(19) United States
(12) Patent Application Publication
(10) Pub. No.: US 2013/0171062 A1
(43) Pub. Date: Jul. 4, 2013

(54) MAPPING IN VIVO EOSINOPHIL ACTIVATION IN EOSINOPHILIC ESOPHAGITIS

(71) Applicants: Leonard Franklin Pease, III, Bountiful, UT (US); Kathryn Peterson, Salt Lake City, UT (US); Gerald J. Gleich, Salt Lake City, UT (US)

(72) Inventors: Leonard Franklin Pease, III, Bountiful, UT (US); Kathryn Peterson, Salt Lake City, UT (US); Gerald J. Gleich, Salt Lake City, UT (US)

(73) Assignee: UNIVERSITY OF UTAH RESEARCH FOUNDATION, Salt Lake City, UT (US)

(21) Appl. No.: 13/639,770

(22) PCT Filed: Apr. 4, 2011
(86) PCT No.: PCT/US11/31098

§ 371 Date: Mar. 15, 2013

Related U.S. Application Data
(60) Provisional application No. 61/320,912, filed on Apr. 5, 2010.

(51) Int. Cl. A61K 51/10

(52) U.S. Cl. CPC A61K 51/1018
USPC 424/1.49; 424/9.1; 424/9.341

(57) ABSTRACT

Provided are compositions and methods for diagnosing eosinophilic esophagitis in a mammalian subject. Further provided are methods of monitoring the course of eosinophilic esophagitis in a subject diagnosed with the disease.

### STATEMENT REGARDING FEDERALLY SPONSORED RESEARCH

This invention was made with government support under NIH grant number R01GM 080784. The government has certain rights in this invention.

### FIELD OF THE INVENTION

This invention relates generally to the field of diagnostics. Specifically, disclosed are compositions and methods for diagnosing and monitoring eosinophilic esophagitis in a subject using labeled antibodies administered to a subject either orally or in an aerosol. Specifically, disclosed are compositions and noninvasive methods for diagnosing and monitoring eosinophilic esophagitis in a subject using labeled antibodies

### BACKGROUND

Eosinophilic esophagitis (EoE) is a chronic disease of the esophagus that affects over 300,000 patients in the U.S. alone. Symptoms include dysphagia (difficulty swallowing liquids or solids or both, >90%), food impaction (solid food sticks in the esophagus, 50%), odynophagia (painful swallowing), heartburn (33%), chest pain, asthma (50%), diarrhea and vomiting (Gonsalves, Kahrilas, Am J Gastroenterol, 2009). The disease primarily occurs in males (75%) in westernized countries with a mean age between 36 and 42 years of age. While present in adults, the disease can also manifest in children. The symptoms of EoE are similar to an atopic allergenic inflammatory condition of the esophagus, affecting up to 10% of adults presenting for upper endoscopy (Mackenzie, Aliment Ther Pharmacol, Gastroenterol, 2008).

Although the source or sources of this disease have not been conclusively identified, investigators have identified several contributing factors. Genetic predisposition may be at work in this disease, at least in part, due to the increased incidence in first degree relatives of EoE patients relative to the general population. Environmental causes may also be important as allergens (i.e., food and aero-allergens) contribute in up to 97% of cases in children (Liacouras, Clin Gastro Hep 2005). Fogg et al (2003) observed worsening of EoE during the pollination season in an allergic patient. Wang et al. (2007) subsequently identified a seasonal variation in identification and severity of the disease in children. Furthermore, Mishra et al. (2001) determined that intranasal administration of Aspergillus fumigatus in a mouse model replicated the esophageal eosinophilic infiltrate seen in EoE. However, EoE is not simply a seasonal allergy of the esophagus. Despite current treatment with swallowed aerosolized steroids, the response rate is little better than 50% (Konikoff, Gastroenterology, 2007).

Food allergies also play an important role, particularly in pediatric EoE. Markowitz et al (2003) found resolution of esophageal eosinophilia after 4 weeks of amino-acid based elemental diet in 49/51 pediatric patients. In the largest analysis to date, Liacouras et al (2005) found a 97% response to an elemental diet in a cohort of 160 children with EoE. However, preliminary data on an elimination diet in adults find less robust responses than those observed in children. The six-food elimination diet (Gonsalves, Dig Dis Week, 2009) demonstrated improvement in 78% but only a 33% resolution rate. Indeed, responses to skin prick testing in adults undergoing food elimination diets suggest a multi-modal (IgE and non-IgE mediated) immunological process, and murine models find both aeroallergens and food each play significant roles (Mishra, J Clin Invest 2001).

In all cases, detection of EoE via a form of endoscopy known as esophagoduodenoscopy (EGD) remains essential, if not sufficient. In this procedure a small tube with a camera on the distal end is passed into the esophagus, stomach, and first portion of the small intestines to visualize the inside of these organs. In EoE, the inflammation occurs in various parts of the esophagus; there is approximately equal incidence of the proximal, distal or both portions of the esophagus being affected (Gangotena, Am J Gastroenterol 2007). EoE also affects the luminal structure of the esophagus. Pronounced rings or furrows can develop into strictures that close off the esophagus, resulting in odynophagia, dysphagia, food impaction and emergency hospital visits. The areas of inflammation are not evenly distributed throughout an affected esophagus, as the disease often presents in patches or select segments of the 25-30 cm long adult esophagus.

Although EGD is a key tool in the identification of EoE, some cases may never present as a “ringed-esophagus” during EGD. Therefore, the only conclusive means currently available to clinicians to positively identify EoE is to detect the presence of eosinophils in biopsy specimens. Tissue samples may be collected during EGD and then examined with traditional histological analysis to confirm or reject a case of EoE. However, the patchy nature of the disease complicates collection of tissue samples for biopsy. For example, biopsies to collect tissue samples are often collected from unaffected areas. For this reason, at least 4 (child) or 5 (adult) biopsy specimens are required to confirm each case of EoE (Gonsalves Gastrointestinal Endosc 2006, Shah Am J Gastroenterol 2009). Furthermore, additional biopsies are required to evaluate the effectiveness of each treatment proposed. This repeated need for endoscopic removal of tissue poses a financial hardship for the patient ($1,500 per visit), and the procedure can be painful, requiring anesthesia.

The key element for diagnosing EoE in a biopsy specimen is the presence of eosinophils. Normal esophageal tissue does not contain eosinophils (Kato et al 1998). These white blood cells were named for their affinity for the red dye eosin. Normally, eosinophils reside in the blood stream, stomach, small and large intestine and lymphatic system (Kato et al, 1998) but infiltrate pathologically into the esophagus in EoE. In biopsy samples, an eosinophil can be identified as a cell 12-17 μm in diameter, a bilobed nucleus and cytoplasmic granules staining red with basic dyes. A tissue count of eosinophils in excess of 15 per field of view at high microscope power indicates EoE. Some clinical evidence suggests that inflammation increases with eosinophil concentration. However, most data suggest that symptoms alone are ineffective in monitoring the disease.

A distinctive characteristic of eosinophils is their granules, each of which is composed of a core and a matrix. The core consists primarily of major basic protein 1 (MBP-1) and the matrix consists of eosinophil peroxidase (EPO), eosinophil derived neurotoxin (EDN) (Peters et al 1986), inter alia. MBP-1 is a highly basic 13.8 kDa protein with 5 unpaired cysteins that accounts for about 55% of the granule's protein (Gleich et al 1974, Gleich et al 1976). It is a member of the C-type lectin family (lectins bind sugars) and has the highest concentration on a per molecule basis (Abu-Ghazaleh et al 1992). EPO has the highest concentration on a per mass basis, while EDN and ECP are members of the RNAse 2 family (Gleich et al 1986). Upon degranulation, an eosinophil releases each of these proteins into the surrounding tissues. Of these, only MBP-1 stimulates histamine release (O'Donnell et al 1983). MBP1 also exfoliates bronchial epithelial cells (Frigas et al 1980) and causes bronchial hyper reactivity (Gundel et al 1991), whereas both MBP-1 and EPO provoke transient bronchial constriction (Gundel et al 1991). These proteins are found in abundance on biopsies in eosinophilic esophagitis (Kephart, Am J Gastroenterol 2010). It is believed that the release of these proteins is responsible for many of the immunogenic responses observed in EoE.

Currently, as symptoms are unable to predict the severity of eosinophilic involvement, the only way to adequately monitor the disease extent and severity is through invasive upper endoscopy with biopsy. Often, in food reintroduction and therapeutic evaluation, this results in several upper endoscopies per year for patients. Due to the cost, invasiveness, and discomfort experienced via this method of monitoring, patients become non-complaint, and, subsequently, the disease is not adequately tracked. Additionally, there is a lack of sensitivity of biopsies in detecting and understanding such a patchy disease—biopsies histologically characterize <0.03% of the entire esophagus, leaving full comprehension about such discontinuous infiltration to one's imagination.

Despite the rapidly growing incidence of EoE, state-of-the-art diagnostic techniques remain inadequate to fully characterize this disease. As such, there exists a need to develop a noninvasive, precise, and comprehensive technique to image and map the distribution of inflammation and deposition of eosinophil granule proteins. Such techniques will provide a tool to diagnose EoE, track disease activity in response to various treatment regimens, and obtain previously unreachable insight into the development and progression of EoE pathophysiology.

### SUMMARY

In accordance with the purposes of this invention, as embodied and broadly described herein, this invention, in one aspect, relates to a method of producing a molecular map of an esophagus in a subject, comprising detecting an eosinophil protein in the lumen of the esophagus in a subject, comprising administering to the subject a labeled antibody directed against an eosinophil protein under conditions wherein the labeled antibody can bind to the eosinophil protein, and detecting a labeled antibody/eosinophil protein complex in the lumen of the esophagus, whereby detecting the labeled antibody/eosinophil protein complex in the lumen of the esophagus produces a molecular map of the esophagus in the subject. The molecular map can be a two- or three-dimensional molecular map.

In another aspect, the invention relates to a method of diagnosing eosinophilic esophagitis in a subject, comprising detecting an eosinophil protein in the lumen of the esophagus in a subject, comprising administering to the subject a labeled antibody directed against an eosinophil protein under conditions wherein the labeled antibody can bind to the eosinophil protein, and detecting a labeled antibody/eosinophil protein complex in the lumen of the esophagus, whereby detecting the labeled antibody/eosinophil protein complex in the lumen of the esophagus diagnoses eosinophilic esophagitis in the subject.

In another aspect, the invention relates to a method of producing a two- or three-dimensional molecular map of an esophagus in a subject, comprising detecting an eosinophil surface protein in the lumen of the esophagus in a subject, comprising administering to the subject a labeled antibody directed against an eosinophil surface protein under conditions wherein the labeled antibody can bind to the eosinophil surface protein, and detecting a labeled antibody/eosinophil surface protein complex in the lumen of the esophagus, whereby detecting the labeled antibody/eosinophil surface protein complex in the lumen of the esophagus produces a molecular map of the esophagus in the subject. The molecular map can be a two- or three-dimensional molecular map.

In another aspect, the invention relates to a method of diagnosing eosinophilic esophagitis in a subject, comprising detecting an eosinophil surface protein in the lumen of the esophagus in a subject, comprising administering to the subject a labeled antibody directed against an eosinophil surface protein under conditions wherein the labeled antibody can bind to the eosinophil surface protein, and detecting a labeled antibody/eosinophil surface protein complex in the lumen of the esophagus, whereby detecting the labeled antibody/eosinophil surface protein complex in the lumen of the esophagus diagnoses eosinophilic esophagitis in the subject.

In another aspect, the invention relates to a method of producing a molecular map of an esophagus in a subject, comprising detecting an eosinophil granule protein in the lumen of the esophagus in a subject, comprising administering to the subject a labeled antibody directed against an eosinophil granule protein under conditions wherein the labeled antibody can bind to the eosinophil granule protein, and detecting a labeled antibody/eosinophil granule protein complex in the lumen of the esophagus, whereby detecting the labeled antibody/eosinophil granule protein complex in the lumen of the esophagus produces a molecular map of the esophagus in the subject. The molecular map can be a two- or three-dimensional molecular map.

In another aspect, the invention relates to a method of diagnosing eosinophilic esophagitis in a subject, comprising detecting an eosinophil granule protein in the lumen of the esophagus in a subject, comprising administering to the subject a labeled antibody directed against an eosinophil granule protein under conditions wherein the labeled antibody can bind to the eosinophil granule protein, and detecting a labeled antibody/eosinophil granule protein complex in the lumen of the esophagus, whereby detecting the labeled antibody/eosinophil granule protein complex in the lumen of the esophagus diagnoses eosinophilic esophagitis in the subject.

In another aspect, the invention relates to a method of producing a molecular map of an esophagus in a subject, comprising detecting a secretory product of an eosinophil in the lumen of the esophagus in a subject, comprising administering to the subject a labeled antibody directed against the secretory product of an eosinophil under conditions wherein the labeled antibody can bind to the secretory product of an eosinophil, and detecting a labeled antibody/a secretory product of an eosinophil complex in the lumen of the esophagus, whereby detecting the labeled antibody/a secretory product of an eosinophil complex in the lumen of the esophagus produces a molecular map of the esophagus in the subject. The molecular map can be a two- or three-dimensional molecular map.

In another aspect, the invention relates to a method of diagnosing eosinophilic esophagitis in a subject, comprising detecting a secretory product of an eosinophil in the lumen of the esophagus in a subject, comprising administering to the subject a labeled antibody directed against the secretory product of an eosinophil under conditions wherein the labeled antibody can bind to the secretory product of an eosinophil, and detecting a labeled antibody/a secretory product of an eosinophil complex in the lumen of the esophagus, whereby detecting the labeled antibody a secretory product of an eosinophil complex in the lumen of the esophagus diagnoses eosinophilic esophagitis in the subject.

In another aspect, the invention relates to a method of producing a molecular map of an esophagus in a subject, comprising detecting an eosinophil protein in the lumen of the esophagus in a subject, comprising administering to the subject a radiolabeled antibody directed against an eosinophil protein under conditions wherein the radiolabeled antibody can bind to the eosinophil protein, and detecting a radiolabeled antibody/eosinophil protein complex in the lumen of the esophagus, whereby detecting the radiolabeled antibody/eosinophil protein complex in the lumen of the esophagus produces a molecular map of the esophagus in the subject. The molecular map can be a two- or three-dimensional molecular map.

In another aspect, the invention relates to a method of diagnosing eosinophilic esophagitis in a subject, comprising detecting an eosinophil protein in the lumen of the esophagus in a subject, comprising administering to the subject a radiolabeled antibody directed against an eosinophil protein under conditions wherein the radiolabeled antibody can bind to the eosinophil protein, and detecting a radiolabeled antibody/eosinophil protein complex in the lumen of the esophagus, whereby detecting the radiolabeled antibody/eosinophil protein complex in the lumen of the esophagus diagnoses eosinophilic esophagitis in the subject.

In another aspect, the invention relates to a method of producing a molecular map of an esophagus in a subject, comprising detecting an eosinophil surface protein in the lumen of the esophagus in a subject, comprising administering to the subject a radiolabeled antibody directed against an eosinophil surface protein under conditions wherein the radiolabeled antibody can bind to the eosinophil surface protein, and detecting a radiolabeled antibody/eosinophil surface protein complex in the lumen of the esophagus, whereby detecting the radiolabeled antibody/eosinophil surface protein complex in the lumen of the esophagus produces a molecular map of the esophagus in the subject. The molecular map can be a two- or three-dimensional molecular map.

In another aspect, the invention relates to a method of diagnosing eosinophilic esophagitis in a subject, comprising detecting an eosinophil surface protein in the lumen of the esophagus in a subject, comprising administering to the subject a radiolabeled antibody directed against an eosinophil surface protein under conditions wherein the radiolabeled antibody can bind to the eosinophil surface protein, and detecting a radiolabeled antibody/eosinophil surface protein complex in the lumen of the esophagus, whereby detecting the radiolabeled antibody/eosinophil surface protein complex in the lumen of the esophagus diagnoses eosinophilic esophagitis in the subject.

In another aspect, the invention relates to a method of producing a molecular map of an esophagus in a subject, comprising detecting an eosinophil granule protein in the lumen of the esophagus in a subject, comprising administering to the subject a radiolabeled antibody directed against an eosinophil granule protein under conditions wherein the radiolabeled antibody can bind to the eosinophil granule protein, and detecting a radiolabeled antibody/eosinophil granule protein complex in the lumen of the esophagus, whereby detecting the radiolabeled antibody/eosinophil granule protein complex in the lumen of the esophagus produces a molecular map of the esophagus in the subject. The molecular map can be a two- or three-dimensional molecular map.

In another aspect, the invention relates to a method of diagnosing eosinophilic esophagitis in a subject, comprising detecting an eosinophil granule protein in the lumen of the esophagus in a subject, comprising administering to the subject a radiolabeled antibody directed against an eosinophil granule protein under conditions wherein the radiolabeled antibody can bind to the eosinophil granule protein, and detecting a radiolabeled antibody/eosinophil granule protein complex in the lumen of the esophagus, whereby detecting the radiolabeled antibody/eosinophil granule protein complex in the lumen of the esophagus diagnoses eosinophilic esophagitis in the subject.

In another aspect, the invention relates to a method of producing a molecular map of an esophagus in a subject, comprising detecting an a secretory product of an eosinophil in the lumen of the esophagus in a subject, comprising administering to the subject a radiolabeled antibody directed against the secretory product of an eosinophil under conditions wherein the radiolabeled antibody can bind to the secretory product of an eosinophil, and detecting a radiolabeled antibody/secretory product of an eosinophil complex in the lumen of the esophagus, whereby detecting the radiolabeled antibody/secretory product of an eosinophil complex in the lumen of the esophagus produces a molecular map of the esophagus in the subject. The molecular map can be a two- or three-dimensional molecular map.

In another aspect, the invention relates to a method of diagnosing eosinophilic esophagitis in a subject, comprising detecting an a secretory product of an eosinophil in the lumen of the esophagus in a subject, comprising administering to the subject a radiolabeled antibody directed against a secretory product of an eosinophil under conditions wherein the radiolabeled antibody can bind to the secretory product of an eosinophil, and detecting a radiolabeled antibody/secretory product of an eosinophil complex in the lumen of the esophagus, whereby detecting the radiolabeled antibody/secretory product of an eosinophil complex in the lumen of the esophagus diagnoses eosinophilic esophagitis in the subject.

In another aspect, relates to a method of producing a molecular map of an esophagus in a subject, comprising detecting an eosinophil protein in the lumen of the esophagus in a subject, comprising administering to the subject a chelated antibody directed against an eosinophil protein under conditions wherein the chelated antibody can bind to the eosinophil protein, and detecting a chelated antibody/eosinophil protein complex in the lumen of the esophagus, whereby detecting the chelated antibody/eosinophil protein complex in the lumen of the esophagus produces a molecular map of the esophagus in the subject. The molecular map can be a two- or three-dimensional molecular map.

In another aspect, the invention relates to a method of diagnosing eosinophilic esophagitis in a subject, comprising detecting an eosinophil protein in the lumen of the esophagus in a subject, comprising administering to the subject a chelated antibody directed against an eosinophil protein under conditions wherein the chelated antibody can bind to the eosinophil protein, and detecting a chelated antibody/eosinophil protein complex in the lumen of the esophagus, whereby detecting the chelated antibody/eosinophil protein complex in the lumen of the esophagus diagnoses eosinophilic esophagitis in the subject.

In another aspect, relates to a method of producing a molecular map of an esophagus in a subject, comprising detecting an eosinophil surface protein in the lumen of the esophagus in a subject, comprising administering to the subject a chelated antibody directed against an eosinophil surface protein under conditions wherein the chelated antibody can bind to the eosinophil surface protein, and detecting a chelated antibody/eosinophil surface protein complex in the lumen of the esophagus, whereby detecting the chelated antibody/eosinophil surface protein complex in the lumen of the esophagus produces a molecular map of the esophagus in the subject. The molecular map can be a two- or three-dimensional molecular map.

In another aspect, the invention relates to a method of diagnosing eosinophilic esophagitis in a subject, comprising detecting an eosinophil surface protein in the lumen of the esophagus in a subject, comprising administering to the subject a chelated antibody directed against an eosinophil surface protein under conditions wherein the chelated antibody can bind to the eosinophil surface protein, and detecting a chelated antibody/eosinophil surface protein complex in the lumen of the esophagus, whereby detecting the chelated antibody/eosinophil surface protein complex in the lumen of the esophagus diagnoses eosinophilic esophagitis in the subject.

In another aspect, relates to a method of producing a molecular map of an esophagus in a subject, comprising detecting an eosinophil granule protein in the lumen of the esophagus in a subject, comprising administering to the subject a chelated antibody directed against an eosinophil granule protein under conditions wherein the chelated antibody can bind to the eosinophil granule protein, and detecting a chelated antibody/eosinophil granule protein complex in the lumen of the esophagus, whereby detecting the chelated antibody/eosinophil granule protein complex in the lumen of the esophagus produces a molecular map of the esophagus in the subject. The molecular map can be a two- or three-dimensional molecular map.

In another aspect, the invention relates to a method of diagnosing eosinophilic esophagitis in a subject, comprising detecting an eosinophil granule protein in the lumen of the esophagus in a subject, comprising administering to the subject a chelated antibody directed against an eosinophil granule protein under conditions wherein the chelated antibody can bind to the eosinophil granule protein, and detecting a chelated antibody/eosinophil granule protein complex in the lumen of the esophagus, whereby detecting the chelated antibody/eosinophil granule protein complex in the lumen of the esophagus diagnoses eosinophilic esophagitis in the subject.

In another aspect, relates to a method of producing a molecular map of an esophagus in a subject, comprising detecting a secretory product of an eosinophil in the lumen of the esophagus in a subject, comprising administering to the subject a chelated antibody directed against a secretory product of an eosinophil under conditions wherein the chelated antibody can bind to the secretory product of an eosinophil, and detecting a chelated antibody/secretory product of an eosinophil complex in the lumen of the esophagus, whereby detecting the chelated antibody/secretory product of an eosinophil complex in the lumen of the esophagus produces a molecular map of the esophagus in the subject. The molecular map can be a two- or three-dimensional molecular map.

In another aspect, the invention relates to a method of diagnosing eosinophilic esophagitis in a subject, comprising detecting a secretory product of an eosinophil in the lumen of the esophagus in a subject, comprising administering to the subject a chelated antibody directed against a secretory product of an eosinophil under conditions wherein the chelated antibody can bind to the secretory product of an eosinophil, and detecting a chelated antibody/secretory product of an eosinophil complex in the lumen of the esophagus, whereby detecting the chelated antibody/secretory product of an eosinophil complex in the lumen of the esophagus diagnoses eosinophilic esophagitis in the subject.

In another aspect, relates to a method of producing a molecular map of an esophagus in a subject, comprising detecting an eosinophil protein in the lumen of the esophagus in a subject, comprising administering to the subject an antibody chelated with a MRI contrast agent directed against an eosinophil protein under conditions wherein the antibody chelated with the MRI contrast agent can bind to the eosinophil protein, and detecting a antibody/eosinophil protein complex in the lumen of the esophagus, whereby detecting the antibody/eosinophil protein complex in the lumen of the esophagus produces a molecular map of the esophagus in the subject. The molecular map can be a two- or three-dimensional molecular map.

In another aspect, the invention relates to a method of diagnosing eosinophilic esophagitis in a subject, comprising detecting an eosinophil protein in the lumen of the esophagus in a subject, comprising administering to the subject an antibody chelated with a MRI contrast agent directed against an eosinophil protein under conditions wherein the an antibody chelated with a MRI contrast agent can bind to the eosinophil protein, and detecting the antibody/eosinophil protein complex in the lumen of the esophagus, whereby detecting the antibody/eosinophil protein complex in the lumen of the esophagus diagnoses eosinophilic esophagitis in the subject.

In another aspect, relates to a method of producing a molecular map of an esophagus in a subject, comprising detecting an eosinophil surface protein in the lumen of the esophagus in a subject, comprising administering to the subject an antibody chelated with a MRI contrast agent directed against an eosinophil surface protein under conditions wherein the antibody chelated with the MRI contrast agent can bind to the eosinophil surface protein, and detecting a antibody/eosinophil surface protein complex in the lumen of the esophagus, whereby detecting the antibody/eosinophil surface protein complex in the lumen of the esophagus produces a molecular map of the esophagus in the subject. The molecular map can be a two- or three-dimensional molecular map.

In another aspect, the invention relates to a method of diagnosing eosinophilic esophagitis in a subject, comprising detecting an eosinophil surface protein in the lumen of the esophagus in a subject, comprising administering to the subject an antibody chelated with a MRI contrast agent directed against an eosinophil surface protein under conditions wherein the an antibody chelated with a MRI contrast agent can bind to the eosinophil surface protein, and detecting the antibody/eosinophil surface protein complex in the lumen of the esophagus, whereby detecting the antibody/eosinophil surface protein complex in the lumen of the esophagus diagnoses eosinophilic esophagitis in the subject.

In another aspect, relates to a method of producing a molecular map of an esophagus in a subject, comprising detecting an eosinophil granule protein in the lumen of the esophagus in a subject, comprising administering to the subject an antibody chelated with a MRI contrast agent directed against an eosinophil granule protein under conditions wherein the antibody chelated with the MRI contrast agent can bind to the eosinophil granule protein, and detecting a antibody/eosinophil granule protein complex in the lumen of the esophagus, whereby detecting the antibody/eosinophil granule protein complex in the lumen of the esophagus produces a molecular map of the esophagus in the subject. The molecular map can be a two- or three-dimensional molecular map.

In another aspect, the invention relates to a method of diagnosing eosinophilic esophagitis in a subject, comprising detecting an eosinophil granule protein in the lumen of the esophagus in a subject, comprising administering to the subject an antibody chelated with a MRI contrast agent directed against an eosinophil granule protein under conditions wherein the an antibody chelated with a MRI contrast agent can bind to the eosinophil granule protein, and detecting the antibody/eosinophil granule protein complex in the lumen of the esophagus, whereby detecting the antibody/eosinophil granule protein complex in the lumen of the esophagus diagnoses eosinophilic esophagitis in the subject.

In another aspect, relates to a method of producing a molecular map of an esophagus in a subject, comprising detecting a secretory product of an eosinophil in the lumen of the esophagus in a subject, comprising administering to the subject an antibody chelated with a MRI contrast agent directed against a secretory product of an eosinophil under conditions wherein the antibody chelated with the MRI contrast agent can bind to the secretory product of an eosinophil, and detecting a antibody/secretory product of an eosinophil complex in the lumen of the esophagus, whereby detecting the antibody/secretory product of an eosinophil complex in the lumen of the esophagus produces a molecular map of the esophagus in the subject. The molecular map can be a two- or three-dimensional molecular map.

In another aspect, the invention relates to a method of diagnosing eosinophilic esophagitis in a subject, comprising detecting a secretory product of an eosinophil in the lumen of the esophagus in a subject, comprising administering to the subject an antibody chelated with a MRI contrast agent directed against a secretory product of an eosinophil under conditions wherein the an antibody chelated with a MRI contrast agent can bind to the secretory product of an eosinophil, and detecting the antibody/secretory product of an eosinophil complex in the lumen of the esophagus, whereby detecting the antibody/secretory product of an eosinophil complex in the lumen of the esophagus diagnoses eosinophilic esophagitis in the subject.

Additional advantages of the invention will be set forth in part in the description which follows, and in part will be obvious from the description, or may be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### BRIEF DESCRIPTION OF THE DRAWINGS

The accompanying drawings, which are incorporated in and constitute a part of this specification, illustrate several embodiments and together with the description illustrate the disclosed compositions and methods.

FIG. 1 shows a chromatograph showing purification of IgG using G-25 sephadex column.

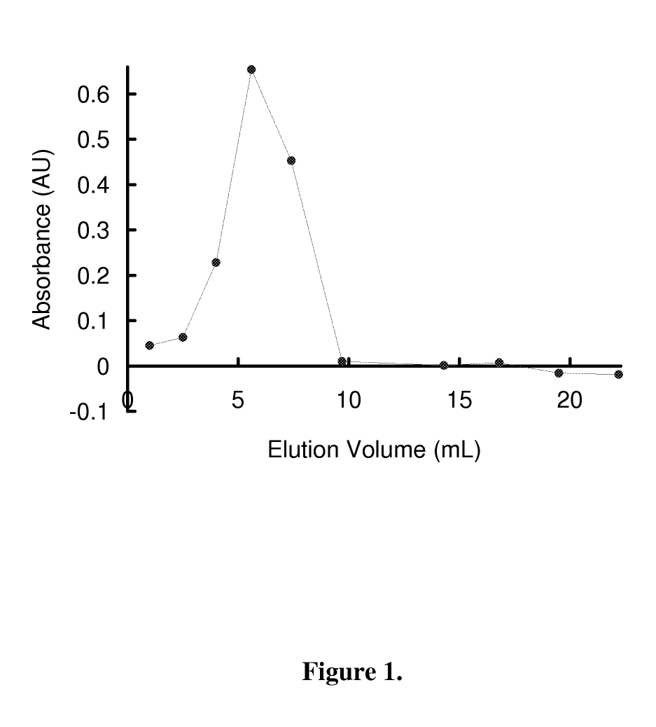

FIG. 2 shows that IgG elutes within the first 3.5 mL of the column, whereas other constituents such as free NHS-MAG3 (second peak in the absorbance curve) or Tc elute later. The amount of Tc label remaining in the column was 276.5 dpm and 181 dpm for Sample 1 and Sample 2 indicating 28% and 37% of the Tc was bound to the antibody, respectively. Thin layer chromatography (TLC) indicated 71.2% binding. Though these are preliminary results and some oxidation of the Sn complex may have occurred, they indicate that this technique can be used to radio label our antibodies as verified by two chromatographic techniques.

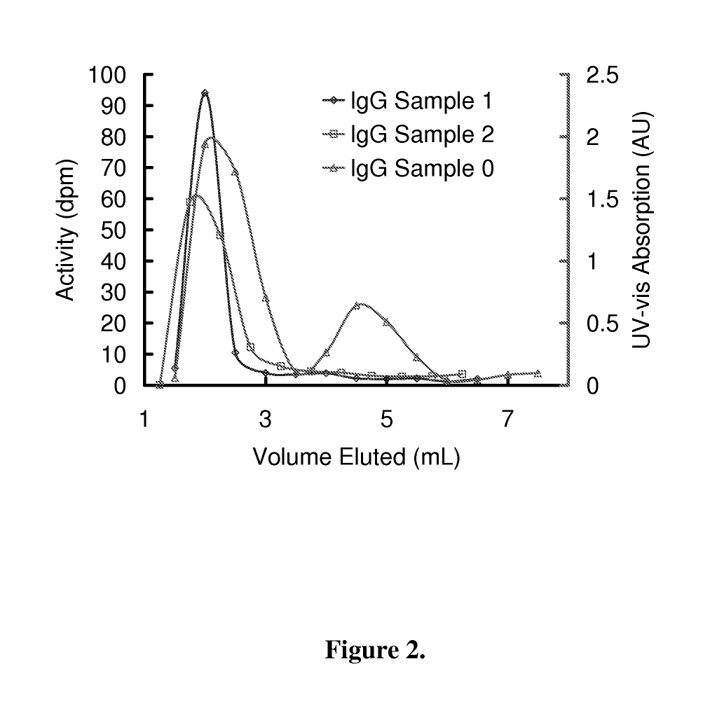

FIG. 3 shows the amount of antibody bound Tc versus incubation time showing that a majority of binding occurs within 30 minutes.

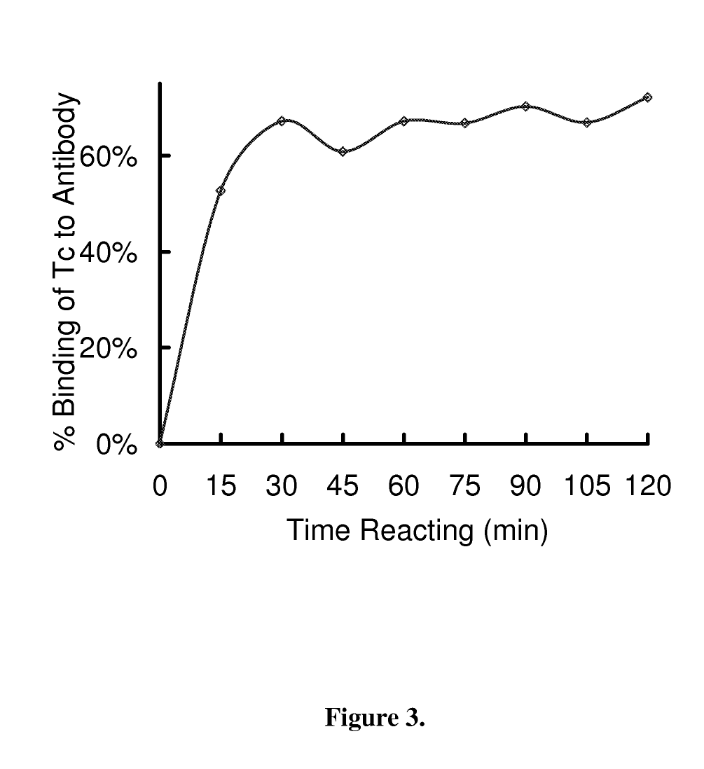

FIG. 4 shows a bar chart showing activity of surface bound species, indicating binding of Tc-MAG3-IgG complex to protein A substrates and the importance of blocking the free Tc.

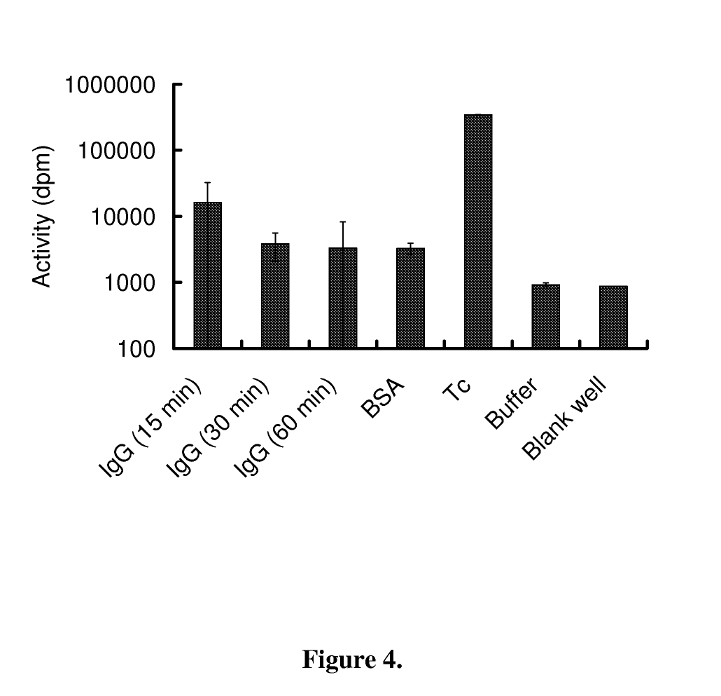

FIG. 5 shows a bar chart showing activity of bound species, indicating binding of Tc-MAG3-IgG complex to protein A substrates and the importance of blocking the free Tc. These results also indicate that protein A may not be the best judge of antibody avidity.

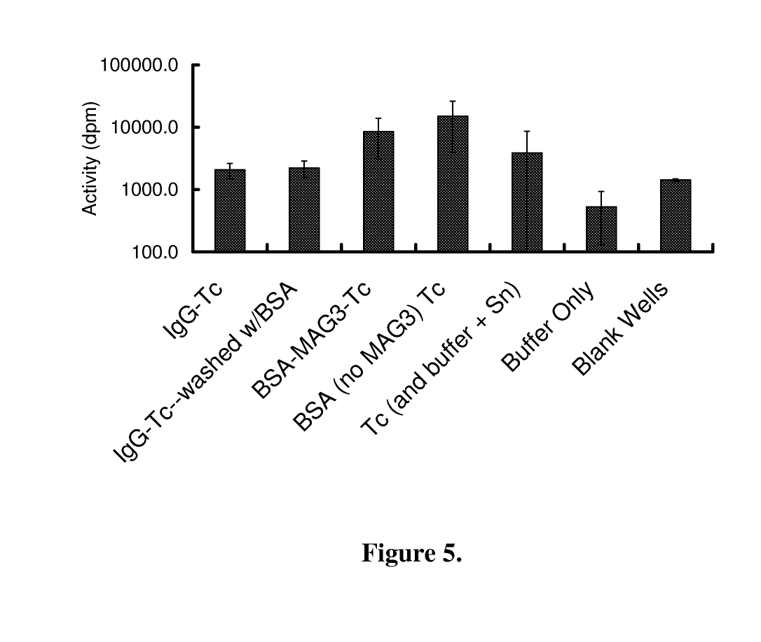

FIG. 6 shows fluorescently stained EoE tissue using antibodies to (a) EDN (b) MB P-1 and (c) negative control at 400×.

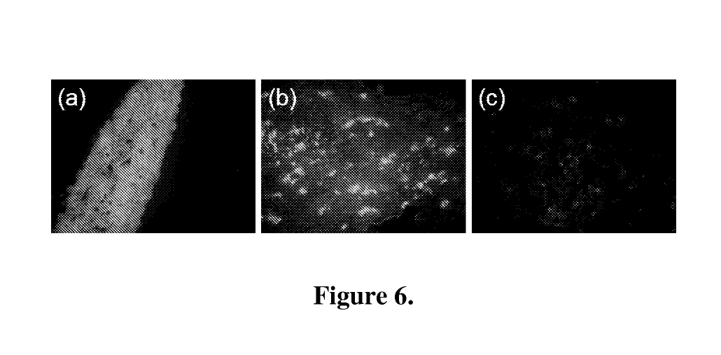

FIG. 7 shows electrospray differential mobility analysis characterization of IgG antibodies on gold nanoparticles.

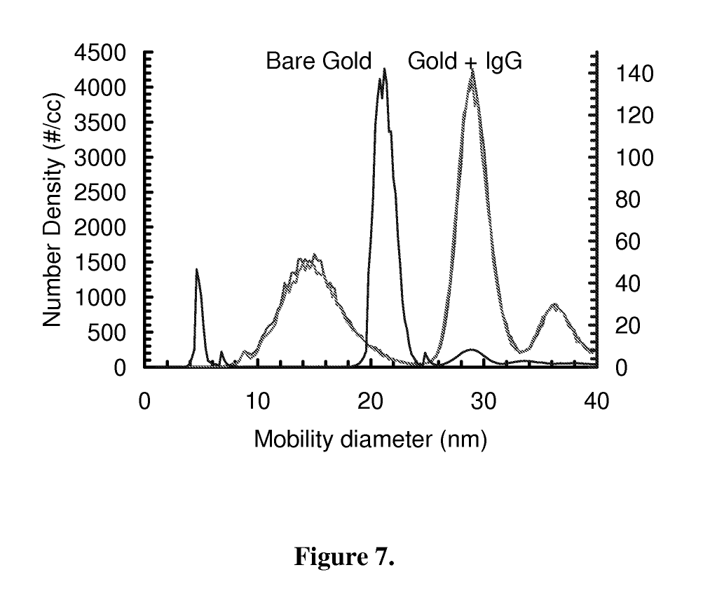

### DETAILED DESCRIPTION

What is needed in the art are compositions and noninvasive methods for diagnosing eosinophilic esophagitis (EoE) in a patient and for monitoring the effectiveness of treatment in the patient in order to decrease patient suffering and cost and to increase patient compliance.

The present invention may be understood more readily by reference to the following detailed description of preferred embodiments of the invention and the Examples included therein and to the Figures and their previous and following description.

Before the present compounds, compositions, articles, devices, and/or methods are disclosed and described, it is to be understood that this invention is not limited to specific synthetic methods, specific labeled antibodies, or antibodies comprising nanoparticles, as such may, of course, vary. It is also to be understood that the terminology used herein is for the purpose of describing particular embodiments only and is not intended to be limiting.

As used in the specification and the appended claims, the singular forms “a,” “an” and “the” include plural referents unless the context clearly dictates otherwise. Thus, for example, reference to “a radiolabeled antibody” or “a labeled antibody” includes mixtures of radiolabeled antibodies or labeled antibodies, respectively; reference to “a nanoparticle” includes mixtures of two or more such nanoparticles, and the like.

Ranges may be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint and independently of the other endpoint.

In this specification and in the claims which follow, reference will be made to a number of terms which shall be defined to have the following meanings:

“Optional” or “optionally” means that the subsequently described event or circumstance may or may not occur, and that the description includes instances where said event or circumstance occurs and instances where it does not. As used herein, by “subject” is meant an individual. Preferably, the subject is a mammal such as a primate, and more preferably a human. The term “subject” includes domesticated animals such as cats, dogs, etc., livestock (e.g., cattle, horses, pigs, sheep, goats, etc.), and laboratory animals (e.g., mice, rabbits, rats, gerbils, guinea pigs, possums, etc.). As used herein, the terms “subject” and “patient” are interchangeable.

Disclosed are compositions and methods for diagnosing EoE in a subject and for monitoring the course of the disease before, during, and after treatment of the disease. Also disclosed herein are compositions and noninvasive methods for diagnosing EoE in a subject and for monitoring the course of the disease before, during, and after treatment of the disease. Thus, provided is a method of producing a three-dimensional map of an esophagus in a subject, comprising detecting an eosinophil protein in the lumen of the esophagus in a subject, comprising administering to the subject a labeled antibody directed against an eosinophil protein under conditions wherein the labeled antibody can bind to the eosinophil protein, and detecting a labeled antibody/eosinophil protein complex in the lumen of the esophagus, whereby detecting the labeled antibody/eosinophil protein complex in the lumen of the esophagus produces a three-dimensional map of the esophagus in the subject.

As used herein, the “lumen of the esophagus” means the mucosal surface of the esophagus, including the squamous epithelium, glandular epithelium, basement membrane, and submucosa.

The term “eosinophil protein” refers to, but is not limited to, eosinophil surface proteins, eosinophil granule proteins, and secretory products of eosinophils.

Proteins on the surface of eosinophils are called eosinophil surface proteins. Examples of eosinophil surface proteins include, but are not limited to, CD9, Eotaxin-3, and CCR3. Proteins that comprise the granules which are released upon activation of eosinophils are called eosinophil granule proteins. Examples of eosinophil granule proteins include, but are not limited to, major basic protein (MBP), major basic protein 1 (MBP-1), major basic protein 2 (MBP-2), eosinophil derived neurotoxin (EDN) (RNase2), eosinophil cationic protein (ECP) (RNase3) and eosinophil peroxidase (EPO). Other examples of eosinophil granule proteins are provided in Kita et al., Biology of Eosinophils, Chapter 19 of Immunology, which is hereby incorporated by reference for its teaching of examples of eosinophil granule proteins. When an eosinophil is activated, granule proteins are released from the cell into the surrounding tissue. The released granule proteins can cause pathologic allergenic responses in the surrounding tissues.

For use in the disclosed methods, provided are antibodies directed against eosinophil proteins including, but is not limited to, eosinophil surface proteins, eosinophil granule proteins, and secretory products of eosinophils. Antibodies to eosinophil proteins can be raised by methods well known in the art. For example, isolated antibodies, antibody fragments and antigen-binding fragments thereof, that specifically bind to CD9, CCR3, MBP-1, MBP-2, EDN, ECP and EPO can be used in the methods described herein. Antibodies that specifically bind MBP-1 are disclosed in Wagner et. al., Placenta 14:671-681, 1993, which is hereby incorporated by reference for its teaching of the same. Such antibodies as disclosed in Wagner et al., Placenta 14:671-681, 1993 can also be used in the disclosed methods. Further provided are antibodies directed against neighboring cells' Eotaxin-3 in the esophagus. Eotaxin-3 is a chemokine that plays a role in the activation of eosinophils.

Also disclosed herein are antibodies that bind secretory products of eosinophils. For example, disclosed herein are antibodies that can bind to secretory products of eosinophils including, but not limited to, Interleukin-1 alpha (IL-1α), Interleukin-2 (IL-2), Interleukin-3 (IL-3), Interleukin-4 (IL-4), Interleukin-5 Interleukin-6 (IL-6), Interleukin-8 (IL-8), Interleukin-10 (IL-10), Interleukin-12 (IL-12), Interleukin-16 (IL-16), Leucotriene B4, Leucotriene C4 (LTC4), Leucotriene C5 (LTC5), 5-HETE, 5, 15- and 8, 15-diHETE, 5-oxo-15-hydroxy-6,8,11,13-ETE, Prostaglandin E1 (PGE1), Prostaglandin E2 (PGE2), 6-Keto-prostaglandin (PGF1), Thromboxane B (TXB2), and Platelet-activating factor (PAF).

Antibodies that bind secretory products of eosinophils can also be used in the methods described herein. For example, provided is a method of producing a three-dimensional map of an esophagus in a subject, comprising detecting a secretory product of eosinophils in the lumen of the esophagus in a subject, comprising administering to the subject a labeled antibody directed against a secretory product of eosinophils under conditions wherein the labeled antibody can bind to the secretory product of eosinophils, and detecting a labeled antibody/secretory product of eosinophil complex in the lumen of the esophagus, whereby detecting the labeled antibody/secretory product of eosinophil complex in the lumen of the esophagus produces a three-dimensional map of the esophagus in the subject.

Also described herein are methods of diagnosing eosinophilic esophagitis in a subject, comprising detecting a secretory product of eosinophils in the lumen of the esophagus in a subject, comprising administering to the subject a labeled antibody directed against a secretory product of eosinophils under conditions wherein the labeled antibody can bind to the secretory product of eosinophils, and detecting a labeled antibody/secretory product of eosinophils complex in the lumen of the esophagus, whereby detecting the labeled antibody/secretory product of eosinophils complex in the lumen of the esophagus diagnoses eosinophilic esophagitis in the subject.

As used herein, the term “antibody” or “antibodies” can refer isolated antibodies, antibody fragments, or antigen-binding fragment thereof. As used herein, the term “antibody” or “antibodies” can also refer to a human antibody or a humanized antibody. Many non-human antibodies (e.g., those derived from mice, rats, or rabbits) are naturally antigenic in humans and, thus, can give rise to undesirable immune responses when administered parenterally to humans. Therefore, the use of human or humanized antibodies in the disclosed methods serves to lessen the chance that an antibody administered to a human will evoke an undesirable immune response.

The term “antibodies” is used herein in a broad sense and includes both polyclonal and monoclonal antibodies. In addition to intact immunoglobulin molecules, disclosed are antibody fragments or polymers of those immunoglobulin molecules, and human or humanized versions of immunoglobulin molecules or fragments thereof, as long as they are chosen for their ability to interact with the target eosinophil proteins disclosed herein.

“Antibody fragments” are portions of a complete antibody. A complete antibody refers to an antibody having two complete light chains and two complete heavy chains. An antibody fragment lacks all or a portion of one or more of the chains. Examples of antibody fragments include, but are not limited to, half antibodies and fragments of half antibodies. A half antibody is composed of a single light chain and a single heavy chain. Half antibodies and half antibody fragments can be produced by reducing an antibody or antibody fragment having two light chains and two heavy chains. Such antibody fragments are referred to as reduced antibodies. Reduced antibodies have exposed and reactive sulfhydryl groups. These sulfhydryl groups can be used as reactive chemical groups for coupling of biomolecules or nanoparticles to the antibody fragment. A preferred half antibody fragment is a F(ab). The hinge region of an antibody or antibody fragment is the region where the light chain ends and the heavy chain goes on.

The term “monoclonal antibody” as used herein refers to an antibody obtained from a substantially homogeneous population of antibodies, i.e., the individual antibodies within the population are identical except for possible naturally occurring mutations that may be present in a small subset of the antibody molecules.

Antibodies suitable for use in the disclosed methods can be grown from hybridomas, purified using affinity chromatography, and characterized for binding by surface plasmon resonance (SPR) against eosinophil surface proteins or eosinophil granule proteins.

Antibodies suitable for use in the disclosed methods can be tested for tissue affinity via assessment of binding to frozen eosinophilic and non-eosinophilic esophageal biopsies.

As used herein, “antibodies” encompasses chimeric antibodies and hybrid antibodies, with dual or multiple antigen or epitope specificities, and fragments, such as F(ab′)2, Fab′, Fab and the like, including hybrid fragments. Thus, fragments of the antibodies that retain the ability to bind their specific antigens are provided. Such antibodies and fragments can be made by techniques known in the art and can be screened for specificity and activity according to the methods set forth in the Examples and in general methods for producing antibodies and screening antibodies for specificity and activity (See Harlow and Lane. Antibodies, A Laboratory Manual. Cold Spring Harbor Publications, New York, (1988)).

Also included within the meaning of “antibody” are conjugates of antibody fragments and antigen binding proteins (single chain antibodies) as described, for example, in U.S. Pat. No. 4,704,692, the contents of which are hereby incorporated by reference.

Optionally, the antibodies are generated in other species and “humanized” for administration in humans. In one aspect, the “humanized” antibody is a human version of the antibody produced by a germ line mutant animal. Humanized forms of non-human (e.g., murine) antibodies are chimeric immunoglobulins, immunoglobulin chains or fragments thereof (such as Fv, Fab, Fab′, F(ab′)2, or other antigen-binding subsequences of antibodies) which contain minimal sequence derived from non-human immunoglobulin. Humanized antibodies include human immunoglobulins (recipient antibody) in which residues from a CDR of the recipient are replaced by residues from a CDR of a non-human species (donor antibody) such as mouse, rat or rabbit having the desired specificity, affinity and capacity. In one embodiment, the present invention provides a humanized version of an antibody, comprising at least one, two, three, four, or up to all CDRs of a monoclonal antibody that specifically binds to a protein or fragment thereof encoded by a gene set forth herein. In some instances, Fv framework residues of the human immunoglobulin are replaced by corresponding non-human residues. Humanized antibodies may also comprise residues that are found neither in the recipient antibody nor in the imported CDR or framework sequences. In general, the humanized antibody can comprise substantially all of or at least one, and typically two, variable domains, in which all or substantially all of the CDR regions correspond to those of a non-human immunoglobulin and all or substantially all of the FR regions are those of a human immunoglobulin consensus sequence. The humanized antibody optimally also can comprise at least a portion of an immunoglobulin constant region (Fc), typically that of a human immunoglobulin (Jones et al., Nature, 321:522-525 (1986); Riechmann et al., Nature, 332:323-327 (1988); and Presta, Curr. Op. Struct. Biol., 2:593-596 (1992)).

Methods for humanizing non-human antibodies are well known in the art. Generally, a humanized antibody has one or more amino acid residues introduced into it from a source that is non-human. These non-human amino acid residues are often referred to as “import” residues, which are typically taken from an “import” variable domain. Humanization can be essentially performed following the method of Winter and co-workers (Jones et al., Nature, 321:522-525 (1986); Riechmann et al., Nature, 332:323-327 (1988); Verhoeyen et al., Science, 239:1534-1536 (1988)), by substituting rodent CDRs or CDR sequences for the corresponding sequences of a human antibody. Accordingly, such “humanized” antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

As used herein, a “label” can include a fluorescent dye, a member of a binding pair, such as biotin/streptavidin, a radiolabel, a metal (e.g., gold), or an epitope tag that can specifically interact with a molecule that can be detected, such as by producing a colored substrate or fluorescence.

Substances suitable for detectably labeling antibodies include fluorescent dyes (also known herein as fluorochromes and fluorophores) and enzymes that react with colorometric substrates (e.g., horseradish peroxidase). The use of fluorescent dyes is generally preferred in the practice of the invention as they can be detected at very low amounts. Furthermore, in the case where multiple antigens are reacted with a single array, each antigen can be labeled with a distinct fluorescent compound for simultaneous detection. Labeled spots on the array are detected using a fluorimeter, the presence of a signal indicating an antigen bound to a specific antibody. Fluorescently labeled antibodies can then be detected in the methods disclosed herein via endoscopy or other known modalities to detect fluorescent labels or dyes.

Fluorophores are compounds or molecules that luminesce. Typically fluorophores absorb electromagnetic energy at one wavelength and emit electromagnetic energy at a second wavelength. Representative fluorophores include, but are not limited to, 1,5 IAEDANS; 1,8-ANS; 4-Methylumbelliferone; 5-carboxy-2,7-dichlorofluorescein; 5-Carboxyfluorescein (5-FAM); 5-Carboxynapthofluorescein; 5-Carboxytetramethylrhodamine (5-TAMRA); 5-Hydroxy Tryptamine (5-HAT); 5-ROX (carboxy-X-rhodamine); 6-Carboxyrhodamine 6G; 6-CR 6G; 6-JOE; 7-Amino-4-methylcoumarin; 7-Aminoactinomycin D (7-AAD); 7-Hydroxy-4-I methylcoumarin; 9-Amino-6-chloro-2-methoxyacridine (ACMA); ABQ; Acid Fuchsin; Acridine Orange; Acridine Red; Acridine Yellow; Acriflavin; Acriflavin Feulgen SITSA; Aequorin (Photoprotein); AFPs—AutoFluorescent Protein—(Quantum Biotechnologies) see sgGFP, sgBFP; Alexa Fluor 350™; Alexa Fluor 430™; Alexa Fluor 488™; Alexa Fluor 532™; Alexa Fluor 546™; Alexa Fluor 568™; Alexa Fluor 594™; Alexa Fluor 633™; Alexa Fluor 647™; Alexa Fluor 660™; Alexa Fluor 680™; Alizarin Complexon; Alizarin Red; Allophycocyanin (APC); AMC, AMCA-S; Aminomethylcoumarin (AMCA); AMCA-X; Aminoactinomycin D; Aminocoumarin; Anilin Blue; Anthrocyl stearate; APC-Cy7; APTRA-BTC; APTS; Astrazon Brilliant Red 4G; Astrazon Orange R; Astrazon Red 6B; Astrazon Yellow 7 GLL; Atabrine; ATTO-TAG™ CBQCA; ATTO-TAG™ FQ; Auramine; Aurophosphine G; Aurophosphine; BAO 9 (Bisaminophenyloxadiazole); BCECF (high pH); BCECF (low pH); Berberine Sulphate; Beta Lactamase; BFP blue shifted GFP (Y66H); Blue Fluorescent Protein; BFP/GFP FRET; Bimane; Bisbenzemide; Bisbenzimide (Hoechst); bis-BTC; Blancophor FFG; Blancophor SV; BOBO™-1; BOBO™-3; Bodipy492/515; Bodipy493/503; Bodipy500/510; Bodipy; 505/515; Bodipy 530/550; Bodipy 542/563; Bodipy 558/568; Bodipy 564/570; Bodipy 576/589; Bodipy 581/591; Bodipy 630/650-X; Bodipy 650/665-X; Bodipy 665/676; Bodipy Fl; Bodipy FL ATP; Bodipy Fl-Ceramide; Bodipy R6G SE; Bodipy TMR; Bodipy TMR-X conjugate; Bodipy TMR-X, SE; Bodipy TR; Bodipy TR ATP; Bodipy TR-X SE; BO-PRO™-1; BO-PRO™-3; Brilliant Sulphoflavin FF; BTC; BTC-5N; Calcein; Calcein Blue; Calcium Crimson-; Calcium Green; Calcium Green-1 Ca²⁺ Dye; Calcium Green-2 Ca²⁺; Calcium Green-5N Ca²⁺; Calcium Green-C18 Ca²⁺; Calcium Orange; Calcofluor White; Carboxy-X-rhodamine (5-ROX); Cascade Blue™; Cascade Yellow; Catecholamine; CCF2 (GeneBlazer); CFDA; CFP (Cyan Fluorescent Protein); CFP/YFP FRET; Chlorophyll; Chromomycin A; Chromomycin A; CL-NERF; CMFDA; Coelenterazine; Coelenterazine cp; Coelenterazine f; Coelenterazine fcp; Coelenterazine h; Coelenterazine hcp; Coelenterazine ip; Coelenterazine n; Coelenterazine O; Coumarin Phalloidin; C-phycocyanine; CPM I Methylcoumarin; CTC; CTC Formazan; Cy2™; Cy3.1 8; Cy3.5™; Cy3™; Cy5.1 8; Cy5.5™; Cy5™; Cy7™; Cyan GFP; cyclic AMP Fluorosensor (FiCRhR); Dabcyl; Dansyl; Dansyl Amine; Dansyl Cadaverine; Dansyl Chloride; Dansyl DHPE; Dansyl fluoride; DAPI; Dapoxyl; Dapoxyl 2; Dapoxyl 3′DCFDA; DCFH (Dichlorodihydrofluorescein Diacetate); DDAO; DHR (Dihydrorhodamine 123); Di-4-ANEPPS; Di-8-ANEPPS (non-ratio); DiA (4-Di 16-ASP); Dichlorodihydrofluorescein Diacetate (DCFH); DiD-Lipophilic Tracer; DiD (DilC18(5)); DIDS; Dihydrorhodamine 123 (DHR); Dil (DilC18(3)); I Dinitrophenol; DiO (DiOC18(3)); DiR; DiR (DilC18(7)); DM-NERF (high pH); DNP; Dopamine; DsRed; DTAF; DY-630-NHS; DY-635-NHS; EBFP; ECFP; EGFP; ELF 97; Eosin; Erythrosin; Erythrosin ITC; Ethidium Bromide; Ethidium homodimer-1 (EthD-1); Euchrysin; EukoLight; Europium (111) chloride; EYFP; Fast Blue; FDA; Feulgen (Pararosaniline); FIF (Formaldehyd Induced Fluorescence); FITC; Flazo Orange; Fluo-3; Fluo-4; Fluorescein (FITC); Fluorescein Diacetate; Fluoro-Emerald; Fluoro-Gold (Hydroxystilbamidine); Fluor-Ruby; Fluor X; FM 1-43™; FM 4-46; Fura Red™ (high pH); Fura Red™/Fluo-3; Fura-2; Fura-2/BCECF; Genacryl Brilliant Red B; Genacryl Brilliant Yellow 10GF; Genacryl Pink 3G; Genacryl Yellow SGF; GeneBlazer; (CCF2); GFP(S65T); GFP red shifted (rsGFP); GFP wild type' non-UV excitation (wtGFP); GFP wild type, UV excitation (wtGFP); GFPuv; Gloxalic Acid; Granular blue; Haematoporphyrin; Hoechst 33258; Hoechst 33342; Hoechst 34580; HPTS; Hydroxycoumarin; Hydroxystilbamidine (FluoroGold); Hydroxytryptamine; Indo-1, high calcium; Indo-1 low calcium; Indodicarbocyanine (DiD); Indotricarbocyanine (DiR); Intrawhite Cf; JC-1; JO JO-1; JO-PRO-1; LaserPro; Laurodan; LDS 751 (DNA); LDS 751 (RNA); Leucophor PAF; Leucophor SF; Leucophor WS; Lissamine Rhodamine; Lissamine Rhodamine B; Calcein/Ethidium homodimer; LOLO-1; LO-PRO-1; Lucifer Yellow; Lyso Tracker Blue; Lyso Tracker Blue-White; Lyso Tracker Green; Lyso Tracker Red; Lyso Tracker Yellow; LysoSensor Blue; LysoSensor Green; LysoSensor Yellow/Blue; Mag Green; Magdala Red (Phloxin B); Mag-Fura Red; Mag-Fura-2; Mag-Fura-5; Mag-lndo-1; Magnesium Green; Magnesium Orange; Malachite Green; Marina Blue; I Maxilon Brilliant Flavin 10 GFF; Maxilon Brilliant Flavin 8 GFF; Merocyanin; Methoxycoumarin; Mitotracker Green FM; Mitotracker Orange; Mitotracker Red; Mitramycin; Monobromobimane; Monobromobimane (mBBr-GSH); Monochlorobimane; MPS (Methyl Green Pyronine Stilbene); NBD; NBD Amine; Nile Red; Nitrobenzoxedidole; Noradrenaline; Nuclear Fast Red; i Nuclear Yellow; Nylosan Brilliant lavin E8G; Oregon Green™; Oregon Green™ 488; Oregon Green™ 500; Oregon Green™ 514; Pacific Blue; Pararosaniline (Feulgen); PBFI; PE-Cy5; PE-Cy7; PerCP; PerCP-Cy5.5; PE-TexasRed (Red 613); Phloxin B (Magdala Red); Phorwite AR; Phorwite BKL; Phorwite Rev; Phorwite RPA; Phosphine 3R; PhotoResist; Phycoerythrin B [PE]; Phycoerythrin R [PE]; PKH26 (Sigma); PKH67; PMIA; Pontochrome Blue Black; POPO-1; POPO-3; PO-PRO-1; PO-I PRO-3; Primuline; Procion Yellow; Propidium lodid (P1); PyMPO; Pyrene; Pyronine; Pyronine B; Pyrozal Brilliant Flavin 7GF; QSY 7; Quinacrine Mustard; Resorufin; RH 414; Rhod-2; Rhodamine; Rhodamine 110; Rhodamine 123; Rhodamine 5 GLD; Rhodamine 6G; Rhodamine B; Rhodamine B 200; Rhodamine B extra; Rhodamine BB; Rhodamine BG; Rhodamine Green; Rhodamine Phallicidine; Rhodamine: Phalloidine; Rhodamine Red; Rhodamine WT; Rose Bengal; R-phycocyanine; R-phycoerythrin (PE); rsGFP; S65A; S65C; S65L; S65T; Sapphire GFP; SBFI; Serotonin; Sevron Brilliant Red 2B; Sevron Brilliant Red 4G; Sevron I Brilliant Red B; Sevron Orange; Sevron Yellow L; sgBFP™ (super glow BFP); sgGFP™ (super glow GFP); SITS (Primuline; Stilbene Isothiosulphonic Acid); SNAFL calcein; SNAFL-1; SNAFL-2; SNARF calcein; SNARF1; Sodium Green; SpectrumAqua; SpectrumGreen; SpectrumOrange; Spectrum Red; SPQ (6-methoxy-N-(3 sulfopropyl) quinolinium); Stilbene; Sulphorhodamine B and C; Sulphorhodamine Extra; SYTO 11; SYTO 12; SYTO 13; SYTO 14; SYTO 15; SYTO 16; SYTO 17; SYTO 18; SYTO 20; SYTO 21; SYTO 22; SYTO 23; SYTO 24; SYTO 25; SYTO 40; SYTO 41; SYTO 42; SYTO 43; SYTO 44; SYTO 45; SYTO 59; SYTO 60; SYTO 61; SYTO 62; SYTO 63; SYTO 64; SYTO 80; SYTO 81; SYTO 82; SYTO 83; SYTO 84; SYTO 85; SYTOX Blue; SYTOX Green; SYTOX Orange; Tetracycline; Tetramethylrhodamine (TRITC); Texas Red™; Texas Red-X™ conjugate; Thiadicarbocyanine (DiSC3); Thiazine Red R; Thiazole Orange; Thioflavin 5; Thioflavin S; Thioflavin TON; Thiolyte; Thiozole Orange; Tinopol CBS (Calcofluor White); TIER; TO-PRO-1; TO-PRO-3; TO-PRO-5; TOTO-1; TOTO-3; TriColor (PE-Cy5); TRITC TetramethylRodaminelsoThioCyanate; True Blue; Tru Red; Ultralite; Uranine B; Uvitex SFC; wt GFP; WW 781; X-Rhodamine; XRITC; Xylene Orange; Y66F; Y66H; Y66W; Yellow GFP; YFP; YO-PRO-1; YO-PRO3; YOYO-1; YOYO-3; Sybr Green; Thiazole orange (interchelating dyes); semiconductor nanoparticles such as quantum dots; or caged fluorophore (which can be activated with light or other electromagnetic energy source), or a combination thereof.

Labeling of an antibody can be either direct or indirect. In direct labeling, the detecting antibody (the antibody for the molecule of interest) or detecting molecule (the molecule that can be bound by an antibody to the molecule of interest) include a label. Detection of the label indicates the presence of the detecting antibody or detecting molecule, which in turn indicates the presence of the molecule of interest or of an antibody to the molecule of interest, respectively. In indirect labeling, an additional molecule or moiety is brought into contact with, or generated at the site of, the immunocomplex. For example, a signal-generating molecule or moiety such as an enzyme can be attached to or associated with the detecting antibody or detecting molecule. The signal-generating molecule can then generate a detectable signal at the site of the immunocomplex. For example, an enzyme, when supplied with suitable substrate, can produce a visible or detectable product at the site of the immunocomplex. ELISAs use this type of indirect labeling.

An antibody used in the disclosed methods can be linked with a suitable radiolabel. Examples of a radiolabel include, but are not limited to, Tc-99m, In-111, I-123, I-131, I-125, Ga-67, Tl-201, Cr-51, and Y-90. Thus, in one aspect, a disclosed antibody can be linked with Tc-99m. In another aspect, a disclosed antibody can be linked to In-111. It is contemplated that a disclosed antibody can be linked to other suitable radiolabels known to persons of ordinary skill in the art. For example, Radiopharmaceuticals in Nuclear Pharmacy and Nuclear Medicine, Second Edition, Richard J. Kowalsky, Steven W. Falen, American Pharmaceutical Association, Washington D.C., 2004 provides examples of other suitable radiolabels. This reference is incorporated herein by reference for its teaching of radiolabels.

As used herein, a “radiolabel” is an isotopic composition that can be attached to a substance to track the substance as it passes through a system. For example, a radiolabel can be attached using any one or more of several commercially available linking agents to antibodies directed against eosinophil proteins including, but not limited to, eosinophil surface proteins, eosinophil granule proteins, or secretory products of eosinophils. Examples of suitable linkers include, but are not limited to, glucoheptonic acid (GHA or GH), diethylenetriaminepentaacetic acid (DTPA), pyrophosphoric acid (PPi), trimetaphosphate, hydroxymethylenediphosphonic acid (HDP), methylenephosphonic acid (MDP). Apcitide ligands, depreotide, dithionite, chelating agents N-hydroxysuccinimidyl S acetylmercaptoacetyltriglycinate (NHS-MAG3), diethylene triamine pentaacetic acid (DTPA), and dimercaptosuccinic acid (DMSA). Other examples of suitable linkers include, but are not limited to, the linkers described in Radiopharmaceuticals in Nuclear Pharmacy and Nuclear Medicine, Second Edition, Richard J. Kowalsky, Steven W. Falen, American Pharmaceutical Association, Washington D.C., 2004 which is hereby incorporated herein by reference for its teaching of the same.

The disclosed antibodies can also be used for MRI. The antibodies can be chelated with a variety of chelating agents including, but not limited to, the linkers disclosed above. The chelator can then be loaded with MRI contrast agents including, but not limited to, gadolinium, iron oxide, or manganese or a combination thereof. Use of MRI for detection of compounds in the esophagus or the lumen of the esophagus has previously been avoided due to the fact that imaging the esophagus with MRI is challenging because the esophagus is so thin. The chelator can also be bound to other contrast agents, including, but not limited to metals to form a chelated antibody.

Labeled antibodies can be purified on a protein G chromatography column, Protein A columns, or gel filtration to separate the labeled protein. Successful linking of the radiolabel to an antibody can be demonstrated by eluting the labeled antibodies from a G chromatography column and surveying for radioactivity.

In another aspect, a disclosed labeled antibody can be attached to a nanoparticle for administration to a subject. A disclosed nanoparticle can comprise (1) an external coating that can specifically target and bind to eosinophil proteins including, but not limited to, eosinophil surface proteins, eosinophil granule proteins, or secretory products of eosinophils and (2) a core that will enhance the contrast of the affected areas. Examples of materials that can comprise the core of a disclosed nanoparticle include, but are not limited to, chitosan, titanium oxide, pluronic/lipiodol, bismuth sulfide, and gold.

In one aspect, a disclosed nanoparticle comprises a gold core. Gold nanoparticles can be safely administered to a subject and provide excellent radio-opacity. A disclosed gold nanoparticle can range in size from about 1.9 nm to about 700 nm. A disclosed gold nanoparticle can also range in size from about 1.9 nm to about 31 nm. Optionally, the surface of a gold nanoparticle can be modified to enhance binding with a disclosed radiolabeled antibody.

A gold nanoparticle can be attached to a disclosed labeled antibody by thiolating protein A or protein G. The thiol is capable of adhering to gold nanoparticles and the protein can bind with the Fc region of the antibody.

Attaching a gold particle to an antibody can be performed by reducing the antibody, so that the two halves of the antibody separate, leaving free thiols. Gold particles can then be introduced which can lead to ligand exchange and binding between the gold and antibody.

A labeled antibody directed against eosinophil proteins including, but not limited to, eosinophil surface proteins, eosinophil granule proteins, secretory products of eosinophils, CCR3, CD9 or Eotaxin-3 can be attached to a nanoparticle and administered to a subject orally or in an aerosol. In one aspect, a disclosed labeled antibody directed against an eosinophil surface protein can be attached to a nanoparticle and then administered to a subject orally or in an aerosol. Thus, for example, a labeled antibody directed against CD9 surface protein can be attached to a gold nanoparticle and administered to a subject orally or in an aerosol. In another example, a labeled antibody directed against CCR3 can be attached to a gold nanoparticle and then administered to a subject orally or in an aerosol. Aerosol compositions can be administered by swallowing a nebulized spray to enter the esophagus similar to nebulizers to distribute bronchodilators into the lungs of asthmatics. To effectively administer the aerosolized radioisotope into the esophagus, the subject can swallow vigorously during aerosol administration. The droplet size can be controlled to allow for better distribution within the esophageal lumen. Mucomyst can be swallowed by the subjects prior to administration of the aerosol and oral dosing to effectively disrupt the esophageal mucous layer. Oral dosing can entail similar ingestion to routine barium studies of the esophagus. The labeled antibodies can be suspended in a thickened mixture (i.e. sucralose) of approximately 15-30 cc volume. The dwell time in the esophagus can be controlled by having the patient lie down with head below feet so that there is some “reflux” or recirculation within the esophagus. In addition, a differential mobility analyzer could be used with electrospray to aerosolize droplets of the material and then the dried material could be sized based on size with the instrument.

In another aspect, a disclosed labeled antibody directed against eosinophil proteins including, but not limited to, eosinophil surface proteins, eosinophil granule proteins, or secretory products of eosinophils can be attached to a nanoparticle and administered to a subject orally or in an aerosol. Thus, for example, a labeled antibody directed against MBP-1 granule proteins can be attached to a gold nanoparticle and then administered to a subject orally or in an aerosol. In another example, a labeled antibody directed against MBP-2 can be attached to a gold nanoparticle and then administered to a subject orally or in an aerosol. In another example, a labeled antibody directed against EDN can be attached to a gold nanoparticle and administered to a subject orally or in an aerosol. In another example, a labeled antibody directed against EPO can be attached to a gold nanoparticle and administered to a subject orally or in an aerosol. In another example, a labeled antibody directed against ECP can be attached to a gold nanoparticle and administered to a subject orally or in an aerosol.

A person of ordinary skill in the art can administer to a subject a composition comprising a mixture of more than one type of labeled antibody, each attached to a gold nanoparticle, in order to enhance the detection of eosinophils in the lumen of the esophagus of a subject. Thus, a disclosed composition can comprise, for example, a mixture of gold nanoparticles attached to labeled antibodies selected from the group of labeled antibodies directed against CD9, CCR3, MBP-1, MBP-2, EDN, Eotaxin-3, ECP and EPO, or any combination thereof.

After administering a composition comprising, for example labeled antibodies directed against eosinophil proteins including, but not limited to, eosinophil surface proteins, eosinophil granule proteins, secretory products of eosinophils or Eotaxin-3, or a mixture thereof, a person of skill can use endoscopy with contrast agents, PET scans, X-ray, conventional or computed tomography (CT), single photon emissions computed tomography (SPECT), endoscopy, or magnetic resonance imaging (MRI) modalities well known in the art to search for and detect the presence of labeled antibody/eosinophil protein complexes adherent to the lumen of the esophagus of a subject where eosinophils have degranulated and caused one or more patches of esophagitis. Optionally, SPECT can be used in combination with MRI and/or CT scans to produce a three-dimensional molecular map of an esophagus having patches of eosinophilic esophagitis. Fiduciary markers on the skin can also be used to position a patient so that they can be imaged from day to day. For example, lasers can be used to position the patient reproducibly. This permits use of multiple scans to be precisely compared. In addition, Gadolinium can be used as a label for MRI scans.

Also disclosed are methods of producing a molecular map of an esophagus in a subject, comprising detecting an eosinophil protein in the lumen of the esophagus in a subject, comprising administering to the subject a labeled antibody directed against an eosinophil protein under conditions wherein the labeled antibody can bind to the eosinophil protein, and detecting a labeled antibody/eosinophil protein complex in the lumen of the esophagus, whereby detecting the labeled antibody/eosinophil protein complex in the lumen of the esophagus produces a molecular map of the esophagus in the subject. The molecular map can be a two- or three-dimensional molecular map.

In another aspect, the invention relates to a method of diagnosing eosinophilic esophagitis in a subject, comprising detecting an eosinophil protein in the lumen of the esophagus in a subject, comprising administering to the subject a labeled antibody directed against an eosinophil protein under conditions wherein the labeled antibody can bind to the eosinophil protein, and detecting a labeled antibody/eosinophil protein complex in the lumen of the esophagus, whereby detecting the labeled antibody/eosinophil protein complex in the lumen of the esophagus diagnoses eosinophilic esophagitis in the subject.

In another aspect, the invention relates to a method of producing a two- or three-dimensional molecular map of an esophagus in a subject, comprising detecting an eosinophil surface protein in the lumen of the esophagus in a subject, comprising administering to the subject a labeled antibody directed against an eosinophil surface protein under conditions wherein the labeled antibody can bind to the eosinophil surface protein, and detecting a labeled antibody/eosinophil surface protein complex in the lumen of the esophagus, whereby detecting the labeled antibody/eosinophil surface protein complex in the lumen of the esophagus produces a molecular map of the esophagus in the subject. The molecular map can be a two- or three-dimensional molecular map.

In another aspect, the invention relates to a method of diagnosing eosinophilic esophagitis in a subject, comprising detecting an eosinophil surface protein in the lumen of the esophagus in a subject, comprising administering to the subject a labeled antibody directed against an eosinophil surface protein under conditions wherein the labeled antibody can bind to the eosinophil surface protein, and detecting a labeled antibody/eosinophil surface protein complex in the lumen of the esophagus, whereby detecting the labeled antibody/eosinophil surface protein complex in the lumen of the esophagus diagnoses eosinophilic esophagitis in the subject.

In another aspect, the invention relates to a method of producing a molecular map of an esophagus in a subject, comprising detecting an eosinophil granule protein in the lumen of the esophagus in a subject, comprising administering to the subject a labeled antibody directed against an eosinophil granule protein under conditions wherein the labeled antibody can bind to the eosinophil granule protein, and detecting a labeled antibody/eosinophil granule protein complex in the lumen of the esophagus, whereby detecting the labeled antibody/eosinophil granule protein complex in the lumen of the esophagus produces a molecular map of the esophagus in the subject. The molecular map can be a two- or three-dimensional molecular map.

In another aspect, the invention relates to a method of diagnosing eosinophilic esophagitis in a subject, comprising detecting an eosinophil granule protein in the lumen of the esophagus in a subject, comprising administering to the subject a labeled antibody directed against an eosinophil granule protein under conditions wherein the labeled antibody can bind to the eosinophil granule protein, and detecting a labeled antibody/eosinophil granule protein complex in the lumen of the esophagus, whereby detecting the labeled antibody/eosinophil granule protein complex in the lumen of the esophagus diagnoses eosinophilic esophagitis in the subject.

In another aspect, the invention relates to a method of producing a molecular map of an esophagus in a subject, comprising detecting a secretory product of an eosinophil in the lumen of the esophagus in a subject, comprising administering to the subject a labeled antibody directed against the secretory product of an eosinophil under conditions wherein the labeled antibody can bind to the secretory product of an eosinophil, and detecting a labeled antibody/a secretory product of an eosinophil complex in the lumen of the esophagus, whereby detecting the labeled antibody/a secretory product of an eosinophil complex in the lumen of the esophagus produces a molecular map of the esophagus in the subject. The molecular map can be a two- or three-dimensional molecular map.

In another aspect, the invention relates to a method of diagnosing eosinophilic esophagitis in a subject, comprising detecting a secretory product of an eosinophil in the lumen of the esophagus in a subject, comprising administering to the subject a labeled antibody directed against the secretory product of an eosinophil under conditions wherein the labeled antibody can bind to the secretory product of an eosinophil, and detecting a labeled antibody/a secretory product of an eosinophil complex in the lumen of the esophagus, whereby detecting the labeled antibody a secretory product of an eosinophil complex in the lumen of the esophagus diagnoses eosinophilic esophagitis in the subject.

In another aspect, the invention relates to a method of producing a molecular map of an esophagus in a subject, comprising detecting an eosinophil protein in the lumen of the esophagus in a subject, comprising administering to the subject a radiolabeled antibody directed against an eosinophil protein under conditions wherein the radiolabeled antibody can bind to the eosinophil protein, and detecting a radiolabeled antibody/eosinophil protein complex in the lumen of the esophagus, whereby detecting the radiolabeled antibody/eosinophil protein complex in the lumen of the esophagus produces a molecular map of the esophagus in the subject. The molecular map produced can be a two- or three-dimensional molecular map. In another aspect, the invention relates to a method of producing a molecular map of an esophagus in a subject, comprising detecting an eosinophil protein in the lumen of the esophagus in a subject, comprising administering to the subject a radiolabeled antibody directed against an eosinophil protein under conditions wherein the radiolabeled antibody can bind to the eosinophil protein, and detecting a radiolabeled antibody/eosinophil protein complex in the lumen of the esophagus, whereby detecting the radiolabeled antibody/eosinophil protein complex in the lumen of the esophagus produces a molecular map of the esophagus in the subject, wherein said radiolabeled antibody/eosinophil protein complex is detected via X-ray analysis.

In another aspect, the invention relates to a method of diagnosing eosinophilic esophagitis in a subject, comprising detecting an eosinophil protein in the lumen of the esophagus in a subject, comprising administering to the subject a radiolabeled antibody directed against an eosinophil protein under conditions wherein the radiolabeled antibody can bind to the eosinophil protein, and detecting a radiolabeled antibody/eosinophil protein complex in the lumen of the esophagus, whereby detecting the radiolabeled antibody/eosinophil protein complex in the lumen of the esophagus diagnoses eosinophilic esophagitis in the subject. In another aspect, the invention relates to a method of diagnosing eosinophilic esophagitis in a subject, comprising detecting an eosinophil protein in the lumen of the esophagus in a subject, comprising administering to the subject a radiolabeled antibody directed against an eosinophil protein under conditions wherein the radiolabeled antibody can bind to the eosinophil protein, and detecting a radiolabeled antibody/eosinophil protein complex in the lumen of the esophagus, whereby detecting the radiolabeled antibody/eosinophil protein complex in the lumen of the esophagus diagnoses eosinophilic esophagitis in the subject, wherein said radiolabeled antibody/eosinophil protein complex is detected via X-ray analysis.

In another aspect, the invention relates to a method of producing a molecular map of an esophagus in a subject, comprising detecting an eosinophil surface protein in the lumen of the esophagus in a subject, comprising administering to the subject a radiolabeled antibody directed against an eosinophil surface protein under conditions wherein the radiolabeled antibody can bind to the eosinophil surface protein, and detecting a radiolabeled antibody/eosinophil surface protein complex in the lumen of the esophagus, whereby detecting the radiolabeled antibody/eosinophil surface protein complex in the lumen of the esophagus produces a molecular map of the esophagus in the subject. The molecular map can be a two- or three-dimensional molecular map. In another aspect, the invention relates to a method of producing a molecular map of an esophagus in a subject, comprising detecting an eosinophil surface protein in the lumen of the esophagus in a subject, comprising administering to the subject a radiolabeled antibody directed against an eosinophil surface protein under conditions wherein the radiolabeled antibody can bind to the eosinophil surface protein, and detecting a radiolabeled antibody/eosinophil surface protein complex in the lumen of the esophagus, whereby detecting the radiolabeled antibody/eosinophil surface protein complex in the lumen of the esophagus produces a molecular map of the esophagus in the subject, wherein said radiolabeled antibody/eosinophil surface protein complex is detected via X-ray analysis.

In another aspect, the invention relates to a method of diagnosing eosinophilic esophagitis in a subject, comprising detecting an eosinophil surface protein in the lumen of the esophagus in a subject, comprising administering to the subject a radiolabeled antibody directed against an eosinophil surface protein under conditions wherein the radiolabeled antibody can bind to the eosinophil surface protein, and detecting a radiolabeled antibody/eosinophil surface protein complex in the lumen of the esophagus, whereby detecting the radiolabeled antibody/eosinophil surface protein complex in the lumen of the esophagus diagnoses eosinophilic esophagitis in the subject. In another aspect, the invention relates to a method of diagnosing eosinophilic esophagitis in a subject, comprising detecting an eosinophil surface protein in the lumen of the esophagus in a subject, comprising administering to the subject a radiolabeled antibody directed against an eosinophil surface protein under conditions wherein the radiolabeled antibody can bind to the eosinophil surface protein, and detecting a radiolabeled antibody/eosinophil surface protein complex in the lumen of the esophagus, whereby detecting the radiolabeled antibody/eosinophil surface protein complex in the lumen of the esophagus diagnoses eosinophilic esophagitis in the subject, wherein said radiolabeled antibody/eosinophil surface protein complex is detected via X-ray analysis.

In another aspect, the invention relates to a method of producing a molecular map of an esophagus in a subject, comprising detecting an eosinophil granule protein in the lumen of the esophagus in a subject, comprising administering to the subject a radiolabeled antibody directed against an eosinophil granule protein under conditions wherein the radiolabeled antibody can bind to the eosinophil granule protein, and detecting a radiolabeled antibody/eosinophil granule protein complex in the lumen of the esophagus, whereby detecting the radiolabeled antibody/eosinophil granule protein complex in the lumen of the esophagus produces a molecular map of the esophagus in the subject. The molecular map can be a two- or three-dimensional molecular map. In another aspect, the invention relates to a method of producing a molecular map of an esophagus in a subject, comprising detecting an eosinophil granule protein in the lumen of the esophagus in a subject, comprising administering to the subject a radiolabeled antibody directed against an eosinophil granule protein under conditions wherein the radiolabeled antibody can bind to the eosinophil granule protein, and detecting a radiolabeled antibody/eosinophil granule protein complex in the lumen of the esophagus, whereby detecting the radiolabeled antibody/eosinophil granule protein complex in the lumen of the esophagus produces a molecular map of the esophagus in the subject, wherein said radiolabeled antibody/eosinophil granule protein complex is detected via X-ray analysis.

In another aspect, the invention relates to a method of diagnosing eosinophilic esophagitis in a subject, comprising detecting an eosinophil granule protein in the lumen of the esophagus in a subject, comprising administering to the subject a radiolabeled antibody directed against an eosinophil granule protein under conditions wherein the radiolabeled antibody can bind to the eosinophil granule protein, and detecting a radiolabeled antibody/eosinophil granule protein complex in the lumen of the esophagus, whereby detecting the radiolabeled antibody/eosinophil granule protein complex in the lumen of the esophagus diagnoses eosinophilic esophagitis in the subject. In another aspect, the invention relates to a method of diagnosing eosinophilic esophagitis in a subject, comprising detecting an eosinophil granule protein in the lumen of the esophagus in a subject, comprising administering to the subject a radiolabeled antibody directed against an eosinophil granule protein under conditions wherein the radiolabeled antibody can bind to the eosinophil granule protein, and detecting a radiolabeled antibody/eosinophil granule protein complex in the lumen of the esophagus, whereby detecting the radiolabeled antibody/eosinophil granule protein complex in the lumen of the esophagus diagnoses eosinophilic esophagitis in the subject, wherein said radiolabeled antibody/eosinophil granule protein complex is detected via X-ray analysis.

In another aspect, the invention relates to a method of producing a molecular map of an esophagus in a subject, comprising detecting an a secretory product of an eosinophil in the lumen of the esophagus in a subject, comprising administering to the subject a radiolabeled antibody directed against the secretory product of an eosinophil under conditions wherein the radiolabeled antibody can bind to the secretory product of an eosinophil, and detecting a radiolabeled antibody/secretory product of an eosinophil complex in the lumen of the esophagus, whereby detecting the radiolabeled antibody/secretory product of an eosinophil complex in the lumen of the esophagus produces a molecular map of the esophagus in the subject. The molecular map can be a two- or three-dimensional molecular map. In another aspect, the invention relates to a method of producing a molecular map of an esophagus in a subject, comprising detecting an a secretory product of an eosinophil in the lumen of the esophagus in a subject, comprising administering to the subject a radiolabeled antibody directed against the secretory product of an eosinophil under conditions wherein the radiolabeled antibody can bind to the secretory product of an eosinophil, and detecting a radiolabeled antibody/secretory product of an eosinophil complex in the lumen of the esophagus, whereby detecting the radiolabeled antibody/secretory product of an eosinophil complex in the lumen of the esophagus produces a molecular map of the esophagus in the subject, wherein said radiolabeled antibody/secretory product of an eosinophil complex is detected via X-ray analysis.

In another aspect, the invention relates to a method of diagnosing eosinophilic esophagitis in a subject, comprising detecting an a secretory product of an eosinophil in the lumen of the esophagus in a subject, comprising administering to the subject a radiolabeled antibody directed against a secretory product of an eosinophil under conditions wherein the radiolabeled antibody can bind to the secretory product of an eosinophil, and detecting a radiolabeled antibody/secretory product of an eosinophil complex in the lumen of the esophagus, whereby detecting the radiolabeled antibody/secretory product of an eosinophil complex in the lumen of the esophagus diagnoses eosinophilic esophagitis in the subject. In another aspect, the invention relates to a method of diagnosing eosinophilic esophagitis in a subject, comprising detecting an a secretory product of an eosinophil in the lumen of the esophagus in a subject, comprising administering to the subject a radiolabeled antibody directed against a secretory product of an eosinophil under conditions wherein the radiolabeled antibody can bind to the secretory product of an eosinophil, and detecting a radiolabeled antibody/secretory product of an eosinophil complex in the lumen of the esophagus, whereby detecting the radiolabeled antibody/secretory product of an eosinophil complex in the lumen of the esophagus diagnoses eosinophilic esophagitis in the subject, wherein said radiolabeled antibody/secretory product of an eosinophil complex is detected via X-ray analysis.

In another aspect, relates to a method of producing a molecular map of an esophagus in a subject, comprising detecting an eosinophil protein in the lumen of the esophagus in a subject, comprising administering to the subject a chelated antibody directed against an eosinophil protein under conditions wherein the chelated antibody can bind to the eosinophil protein, and detecting a chelated antibody/eosinophil protein complex in the lumen of the esophagus, whereby detecting the chelated antibody/eosinophil protein complex in the lumen of the esophagus produces a molecular map of the esophagus in the subject. The molecular map can be a two- or three-dimensional molecular map. In a preferred embodiment, the chelated antibodies are detected via MRI analysis.

In another aspect, the invention relates to a method of diagnosing eosinophilic esophagitis in a subject, comprising detecting an eosinophil protein in the lumen of the esophagus in a subject, comprising administering to the subject a chelated antibody directed against an eosinophil protein under conditions wherein the chelated antibody can bind to the eosinophil protein, and detecting a chelated antibody/eosinophil protein complex in the lumen of the esophagus, whereby detecting the chelated antibody/eosinophil protein complex in the lumen of the esophagus diagnoses eosinophilic esophagitis in the subject.

In another aspect, relates to a method of producing a molecular map of an esophagus in a subject, comprising detecting an eosinophil surface protein in the lumen of the esophagus in a subject, comprising administering to the subject a chelated antibody directed against an eosinophil surface protein under conditions wherein the chelated antibody can bind to the eosinophil surface protein, and detecting a chelated antibody/eosinophil surface protein complex in the lumen of the esophagus, whereby detecting the chelated antibody/eosinophil surface protein complex in the lumen of the esophagus produces a molecular map of the esophagus in the subject. The molecular map can be a two- or three-dimensional molecular map. In a preferred embodiment, the chelated antibodies are detected via MRI analysis.

In another aspect, the invention relates to a method of diagnosing eosinophilic esophagitis in a subject, comprising detecting an eosinophil surface protein in the lumen of the esophagus in a subject, comprising administering to the subject a chelated antibody directed against an eosinophil surface protein under conditions wherein the chelated antibody can bind to the eosinophil surface protein, and detecting a chelated antibody/eosinophil surface protein complex in the lumen of the esophagus, whereby detecting the chelated antibody/eosinophil surface protein complex in the lumen of the esophagus diagnoses eosinophilic esophagitis in the subject. In a preferred embodiment, the chelated antibodies are detected via MRI analysis.

In another aspect, relates to a method of producing a molecular map of an esophagus in a subject, comprising detecting an eosinophil granule protein in the lumen of the esophagus in a subject, comprising administering to the subject a chelated antibody directed against an eosinophil granule protein under conditions wherein the chelated antibody can bind to the eosinophil granule protein, and detecting a chelated antibody/eosinophil granule protein complex in the lumen of the esophagus, whereby detecting the chelated antibody/eosinophil granule protein complex in the lumen of the esophagus produces a molecular map of the esophagus in the subject. The molecular map can be a two- or three-dimensional molecular map. In a preferred embodiment, the chelated antibodies are detected via MRI analysis.

In another aspect, the invention relates to a method of diagnosing eosinophilic esophagitis in a subject, comprising detecting an eosinophil granule protein in the lumen of the esophagus in a subject, comprising administering to the subject a chelated antibody directed against an eosinophil granule protein under conditions wherein the chelated antibody can bind to the eosinophil granule protein, and detecting a chelated antibody/eosinophil granule protein complex in the lumen of the esophagus, whereby detecting the chelated antibody/eosinophil granule protein complex in the lumen of the esophagus diagnoses eosinophilic esophagitis in the subject. In a preferred embodiment, the chelated antibodies are detected via MRI analysis.

In another aspect, relates to a method of producing a molecular map of an esophagus in a subject, comprising detecting a secretory product of an eosinophil in the lumen of the esophagus in a subject, comprising administering to the subject a chelated antibody directed against a secretory product of an eosinophil under conditions wherein the chelated antibody can bind to the secretory product of an eosinophil, and detecting a chelated antibody/secretory product of an eosinophil complex in the lumen of the esophagus, whereby detecting the chelated antibody/secretory product of an eosinophil complex in the lumen of the esophagus produces a molecular map of the esophagus in the subject. The molecular map can be a two- or three-dimensional molecular map. In a preferred embodiment, the chelated antibodies are detected via MRI analysis.

In another aspect, the invention relates to a method of diagnosing eosinophilic esophagitis in a subject, comprising detecting a secretory product of an eosinophil in the lumen of the esophagus in a subject, comprising administering to the subject a chelated antibody directed against a secretory product of an eosinophil under conditions wherein the chelated antibody can bind to the secretory product of an eosinophil, and detecting a chelated antibody/secretory product of an eosinophil complex in the lumen of the esophagus, whereby detecting the chelated antibody/secretory product of an eosinophil complex in the lumen of the esophagus diagnoses eosinophilic esophagitis in the subject. In a preferred embodiment, the chelated antibodies are detected via MRI analysis.

In another aspect, relates to a method of producing a molecular map of an esophagus in a subject, comprising detecting an eosinophil protein in the lumen of the esophagus in a subject, comprising administering to the subject an antibody chelated with a MRI contrast agent directed against an eosinophil protein under conditions wherein the antibody chelated with the MRI contrast agent can bind to the eosinophil protein, and detecting a antibody/eosinophil protein complex in the lumen of the esophagus, whereby detecting the antibody/eosinophil protein complex in the lumen of the esophagus produces a molecular map of the esophagus in the subject. The molecular map can be a two- or three-dimensional molecular map. In a preferred embodiment, the antibodies chelated with a MRI contrast agent are detected via MRI analysis.

In another aspect, the invention relates to a method of diagnosing eosinophilic esophagitis in a subject, comprising detecting an eosinophil protein in the lumen of the esophagus in a subject, comprising administering to the subject an antibody chelated with a MRI contrast agent directed against an eosinophil protein under conditions wherein the an antibody chelated with a MRI contrast agent can bind to the eosinophil protein, and detecting the antibody/eosinophil protein complex in the lumen of the esophagus, whereby detecting the antibody/eosinophil protein complex in the lumen of the esophagus diagnoses eosinophilic esophagitis in the subject. In a preferred embodiment, the antibodies chelated with a MRI contrast agent are detected via MRI analysis.

In another aspect, relates to a method of producing a molecular map of an esophagus in a subject, comprising detecting an eosinophil surface protein in the lumen of the esophagus in a subject, comprising administering to the subject an antibody chelated with a MRI contrast agent directed against an eosinophil surface protein under conditions wherein the antibody chelated with the MRI contrast agent can bind to the eosinophil surface protein, and detecting a antibody/eosinophil surface protein complex in the lumen of the esophagus, whereby detecting the antibody/eosinophil surface protein complex in the lumen of the esophagus produces a molecular map of the esophagus in the subject. The molecular map can be a two- or three-dimensional molecular map. In a preferred embodiment, the antibodies chelated with a MRI contrast agent are detected via MRI analysis.

In another aspect, the invention relates to a method of diagnosing eosinophilic esophagitis in a subject, comprising detecting an eosinophil surface protein in the lumen of the esophagus in a subject, comprising administering to the subject an antibody chelated with a MRI contrast agent directed against an eosinophil surface protein under conditions wherein the an antibody chelated with a MRI contrast agent can bind to the eosinophil surface protein, and detecting the antibody/eosinophil surface protein complex in the lumen of the esophagus, whereby detecting the antibody/eosinophil surface protein complex in the lumen of the esophagus diagnoses eosinophilic esophagitis in the subject. In a preferred embodiment, the antibodies chelated with a MRI contrast agent are detected via MRI analysis.

In another aspect, relates to a method of producing a molecular map of an esophagus in a subject, comprising detecting an eosinophil granule protein in the lumen of the esophagus in a subject, comprising administering to the subject an antibody chelated with a MRI contrast agent directed against an eosinophil granule protein under conditions wherein the antibody chelated with the MRI contrast agent can bind to the eosinophil granule protein, and detecting a antibody/eosinophil granule protein complex in the lumen of the esophagus, whereby detecting the antibody/eosinophil granule protein complex in the lumen of the esophagus produces a molecular map of the esophagus in the subject. The molecular map can be a two- or three-dimensional molecular map. In a preferred embodiment, the antibodies chelated with a MRI contrast agent are detected via MRI analysis.

In another aspect, the invention relates to a method of diagnosing eosinophilic esophagitis in a subject, comprising detecting an eosinophil granule protein in the lumen of the esophagus in a subject, comprising administering to the subject an antibody chelated with a MRI contrast agent directed against an eosinophil granule protein under conditions wherein the an antibody chelated with a MRI contrast agent can bind to the eosinophil granule protein, and detecting the antibody/eosinophil granule protein complex in the lumen of the esophagus, whereby detecting the antibody/eosinophil granule protein complex in the lumen of the esophagus diagnoses eosinophilic esophagitis in the subject. In a preferred embodiment, the antibodies chelated with a MRI contrast agent are detected via MRI analysis.

In another aspect, relates to a method of producing a molecular map of an esophagus in a subject, comprising detecting a secretory product of an eosinophil in the lumen of the esophagus in a subject, comprising administering to the subject an antibody chelated with a MRI contrast agent directed against a secretory product of an eosinophil under conditions wherein the antibody chelated with the MRI contrast agent can bind to the secretory product of an eosinophil, and detecting a antibody/secretory product of an eosinophil complex in the lumen of the esophagus, whereby detecting the antibody/secretory product of an eosinophil complex in the lumen of the esophagus produces a molecular map of the esophagus in the subject. The molecular map can be a two- or three-dimensional molecular map. In a preferred embodiment, the antibodies chelated with a MRI contrast agent are detected via MRI analysis.

In another aspect, the invention relates to a method of diagnosing eosinophilic esophagitis in a subject, comprising detecting a secretory product of an eosinophil in the lumen of the esophagus in a subject, comprising administering to the subject an antibody chelated with a MRI contrast agent directed against a secretory product of an eosinophil under conditions wherein the an antibody chelated with a MRI contrast agent can bind to the secretory product of an eosinophil, and detecting the antibody/secretory product of an eosinophil complex in the lumen of the esophagus, whereby detecting the antibody/secretory product of an eosinophil complex in the lumen of the esophagus diagnoses eosinophilic esophagitis in the subject. In a preferred embodiment, the antibodies chelated with a MRI contrast agent are detected via MRI analysis.

Disclosed herein are methods of diagnosing eosinophilic esophagitis in a subject, comprising detecting an eosinophil protein in the lumen of the esophagus in a subject, comprising administering to the subject a labeled antibody directed against an eosinophil protein under conditions wherein the labeled antibody can bind to the eosinophil protein, and detecting a labeled antibody/eosinophil protein complex in the lumen of the esophagus, whereby detecting the labeled antibody/eosinophil protein complex in the lumen of the esophagus diagnoses eosinophilic esophagitis in the subject. In some embodiments, one or more eosinophil proteins can be detected simultaneously or nonconcurrently in the methods described herein. In such embodiments, multiple labeled antibodies can be used to form multiple labeled antibody/eosinophil protein complexes, where the label on each antibody can be the same or different.

Also provided is a method of detecting and monitoring change in eosinophilic esophagitis in a subject diagnosed with eosinophilic esophagitis, comprising: a) producing a first three-dimensional map of the esophagus in a subject diagnosed with eosinophilic esophagitis according to the methods disclosed herein, b) producing a second three-dimensional map of the esophagus in the subject at a later time, and c) comparing the second three-dimensional map with the first three-dimensional map, whereby detecting a change in the second three-dimensional map compared to the first three-dimensional map detects and monitors change in eosinophilic esophagitis in the subject.

Also described are methods of detecting eosinophilic degranulation in a subject, comprising detecting a granule protein in a subject, comprising administering to the subject a radiolabeled antibody directed against a granule protein under conditions wherein the radiolabeled antibody can bind to the granule protein, and detecting a radiolabeled antibody/granule protein complex, whereby detecting the radiolabeled antibody/granule protein complex indicates the presence of eosinophilic degranulation in the subject. For example, the granule protein can be MBP-1, ECP, EDN, or EPO.

Also described are methods of detecting eosinophilic degranulation in a subject, comprising detecting a granule protein in a subject, comprising administering to the subject a radiolabeled antibody directed against a granule protein under conditions wherein the radiolabeled antibody can bind to the granule protein, and detecting a radiolabeled antibody/granule protein complex, whereby detecting the radiolabeled antibody/granule protein complex indicates the presence of eosinophilic degranulation in the subject. The methods described herein can further comprise detecting eosinophils in the subject, comprising detecting an eosinophilic protein in a subject, comprising administering to the subject a radiolabeled antibody directed against an eosinophilic protein under conditions wherein the radiolabeled antibody can bind to the eosinophilic protein, and detecting a radiolabeled antibody/eosinophilic protein complex, whereby detecting the radiolabeled antibody/eosinophilic protein complex indicates the presence of eosinophils in the subject.

Also described are methods of detecting eosinophilic degranulation in a subject, comprising detecting at least one granule protein in a subject, comprising administering to the subject a labeled antibody directed against a granule protein under conditions wherein the labeled antibody can bind to the granule protein, and detecting a labeled antibody/granule protein complex, whereby detecting the labeled antibody/granule protein complex indicates the presence of eosinophilic degranulation in the subject, wherein the label for the labeled antibody directed against a granule protein and the label for the labeled antibody directed against an eosinophilic protein are different.

Also described are methods of detecting eosinophilic degranulation in a subject, comprising detecting multiple granule proteins in a subject, comprising administering to the subject a labeled antibody directed against a granule protein under conditions wherein the labeled antibody can bind to the granule protein, and detecting a labeled antibody/granule protein complex, whereby detecting the labeled antibody/granule protein complex indicates the presence of eosinophilic degranulation in the subject, wherein the label for the labeled antibody directed against a granule protein and the label for the labeled antibody directed against an eosinophilic protein are different. In certain embodiments, multiple antibodies can be used to detect multiple granule proteins. Such antibodies can be the same or different antibodies. In addition, the multiple granule proteins can be different or the same. In some embodiments, one or more granule proteins can be detected simultaneously or nonconcurrently.

The methods described herein can include nebulization of labeled antibodies into the lung (e.g. for localization in asthma) and injection intravenously (e.g. for localization throughout the body in the hypereosinophilic syndrome, eosinophil-associated intestinal disease, cardiac diseases, pulmonary diseases, fibrosing diseases, thyroid disease, central nervous system diseases, kidney and bladder diseases and liver diseases).

In addition, nebulized antibodies can be used in the methods described herein. For example, nebulized antibodies can be used to identify eosinophil associated asthma or intravenously injected antibodies can be used to identify eosinophils at other sites in the body in the course of these eosinophil-associated diseases.

Presently, identification of eosinophil infiltration depends on biopsy and counting of eosinophils in tissues and that can be difficult. For example, patients suspected to have infiltration and degranulation of eosinophils in the heart must undergo a biopsy of the heart tissues from the left and right ventricles (and these biopsies will be stained for intact eosinophils and for degranulation). In order to improve on these methods the labeled antibodies described herein can be used for EoE for lung and for systemic administration. For example, the antibodies described herein can be administered by nebulization or intravenous injection. For example, nebulization can be used to deliver the antibodies described herein to patients with asthma who have eosinophil lung infiltration. Nebulization of the labeled antibodies can permit identification of eosinophil lung infiltration.

Also disclosed herein are methods comprising injecting the antibodies described herein to localize eosinophils in other tissues known to comprise eosinophils.

A person of skill can choose to follow and monitor a subject by producing additional subsequent three-dimensional maps over time without prescribing treatment to learn the natural course of eosinophilic esophagitis in the subject. Alternatively, a person of skill can monitor the effectiveness of therapy in a subject diagnosed with eosinophilic esophagitis by producing a series of three-dimensional maps of the subject's esophagus over time and comparing subsequent maps to previous maps to look for resolution of patches of esophagitis. For example, three-dimensional maps can be generated every day over a 3 day period or every three days over a one week period. Scans can be utilized to determine onset/presence of disease (resolution of disease is known—from prior endoscopic studies—to take over 2 weeks). Thus, in some instances, intervals between scans can be no more frequent than every 2 days.

In addition, the location of the full length of the esophagus using barium fluoroscopy can be used in combination with the methods described herein. The barium swallow is well established and can effectively outline the location of the esophagus. Barium fluoroscopy can be used prior to, at the same time as, or after the methods described herein.

### EXAMPLES

The following examples are put forth so as to provide those of ordinary skill in the art with a complete disclosure and description of how the compounds, compositions, articles, devices and/or methods claimed herein are made and evaluated, and are intended to be purely exemplary of the invention and are not intended to limit the scope of what the inventors regard as their invention. Efforts have been made to ensure accuracy with respect to numbers (e.g., amounts, temperature, etc.), but some errors and deviations should be accounted for. Unless indicated otherwise, parts can be parts by mole, temperature is in ° C. or is at ambient temperature, and pressure is at or near atmospheric.

### Example 1

Goat polyclonal anti-MBP-1 (SC-34012) was purchased from Santa Cruz Biotechnology, Inc. (Santa Cruz, Calif., USA). The antibodies were stored in 4° C. refrigeration until use. The antibodies were purified using a G25 Sephadex column (HiTrap Desalting Column, Pierce). The antibody concentration was confirmed using UV-vis spectroscopy.

To demonstrate that antibodies can be radiolabeled, polyclonal IgG protein (Sigma) was labeled with the chelator, N-hydroxysuccinimidyl S-acetylmercaptoacetyltriglycinate (NHS-MAG3) as described by Wang, et al. Nat Protoc, 2007. 2(4): p. 972-8. IgG protein (dissolved in 0.3 M HEPES to a concentration of 18 mg/mL) was combined with NHS-MAG3 powder at a molar ratio of 1:15 (antibody:NHS-MAG3). After 2 hours of incubation, chelated antibody was separated using a G25 Sephadex column (HiTrap Desalting Column, Pierce). The final concentration of collected fractions was determined by UV-vis.

Radionuclide Tc-99m was added to the chelated antibodies using the procedure adopted from Wang, et al. Nat Protoc, 2007. 2(4): p. 972-8. Forty micrograms of MAG3-protein conjugate was added to 45 uL of 0.25 M ammonium acetate and 15 uL tartrate buffer and vortexed. Then 25 uL of Tc-99m and 5 uL of Sn solution (4 mg/mL of SnCl₂.2H₂O in tartrate buffer free of stannic chloride) was added and gently vortexed before incubating for 1 hour at ambient. Binding efficacy was assessed using a Sephadex G-25 (GE Lifesciences) column. The radioactivity of each aliquot and the column was measured using a dose calibrator and plotted. The labeling efficiency was assessed by chromatography using ITLC developed in 0.9% saline. After chelating technetium, the results showed that Ab-MAG3-Tc complex forms in <30 min.

Tissue specimens were collected from patients undergoing clinical upper endoscopy. Biopsies approximately 1-2 mm in diameter can be taken from one or more places in the lower esophagus (5 cm proximal to the gastro-esophageal junction) and repeated 10 cm proximal to the initial site in the proximal esophagus. Tissue samples were taken via sterile forceps for frozen sectioning. Samples were immediately wrapped in aluminum foil and placed in liquid nitrogen. After the sampling is complete, the samples were then transferred immediately to a −80° C. freezer where they were kept until sectioning. The samples were stored at −80° C. until use. Aliquots of the radiolabeled antibody were added to the tissue. After washing in triplicate, the counts were measured using a dose calibrator and plotted.

### Example 2

### Detection of Radiolabeled Esophagectomy using SPECT/MRI or SPECT/CT

To demonstrate γ-camera imaging of the biopsy/radiolabeled antibody complex, a surrogate of an EoE-involved esophagus can be used. Cadaveric esophagus can be implanted with 20-30 biopsy specimens from patients with EoE. The first surrogates can be prepared by fixing freshly obtained (IRB 00044645) biopsy samples with superglue to the internal surface of a cooled cadaveric esophagus. Radiolabeled antibodies can be prepared as described herein. Ideally 10 uCi per biopsy can be available for detection. Clip lower end of tissue lumen. Pour in antibodies. Incubate for an appropriate amount of time that will be the same as for human samples. Pour out antibody solution. Image with combination SPECT/MRI or SPECT/CT. NIH's Image J program can be used to determine the intensity of the exposed spots relative to background. The intensity of the spot can be correlated with the eosinophil concentration. All experiments can be run in triplicate except where noted. The data can be represented with mean and standard deviation. Statistical differences can be analyzed using one-way analysis of variance (ANOVA) with the Tukey-Kramer post-hoc test, considering p<0.05 to be significant.

### Example 3

### Antibody Generation, Characterization, and Purification

Murine monoclonal anti-CD-9 (SC-13118-FITC), and goat polyclonal anti-Eotaxin-3 (SC-19353), were purchased from Santa Cruz Biotechnology, Inc. (Santa Cruz, Calif., USA). The anti-CD9 antibody is labeled with FITC. A rabbit anti-CCR3 polyclonal antibody was purchased from GenScript (A00977). A murine monoclonal anti-EDN (J167-6C5) antibody at a concentration of 2.1 mg/mL in sodium azide was prepared from hybridomas. Panels of monoclonal antibodies to MBP-1 (>20 hybridomas), EPO (5 hybridomas) and EDN (>5 hybridomas) were reviewed. The hybridomas producing these antibodies can be grown in 4 L spinner flasks. The monoclonal antibodies can then be harvested from hybridoma supernatants by protein G affinity chromatography using established procedures. The antibodies can be stored in 4° C. refrigeration until use.

The antibodies can then be purified from supernatants by affinity chromatography using protein G or protein A columns.

ES-DMA-CPC analysis of antibodies provides a tool to determine their purity and aggregation state as described in detail by Pease, et al. Briefly, solutions containing antibodies were electrosprayed (TSI, Inc., Shore View, Minn., #3480) to produce a narrow distribution of droplet diameters (Kaufman, 2000). Aerosolized droplets were produced only under conditions where a stable cone-jet meniscus at the tip of a 25 μm inner diameter capillary was visually observed. Immediately downstream of the ES, droplets pass through a neutralizing chamber containing a Po-210 ionizing radiation source from which a majority of droplets emerge with a charge of +1, 0, or −1. As the droplets evaporated, this charge remained on the dried proteins. These proteins pass into the differential mobility analyzer (DMA; TSI, Inc., #3080). The DMA acts like an ion-mobility band-pass filter that for a given electrode voltage and gas flow rate enables a narrow size band of ions to be sent to the condensation particle counter (CPC), which measures the number concentration of particles in the gas (TSI, Inc., #3025A). In this manner proteins with diameters greater than 3 nm can be sized. The sampling and DMA sheath flow rates were 1.2 and 30 L/min, respectively. The conversion from voltage to mobility size has been described in detail elsewhere (Mulholland et al., 2006; Pease et al., 2007). Because a negative bias was applied to ions within the DMA, only proteins that acquire a positive charge are detected. The fraction of proteins emerging with a positive charge is size dependent. A modified expression for the Boltzmann distribution (Wiedensohler, 1988) was used to correct for this effect, transforming the distribution of positively charged proteins into the complete distribution of all proteins regardless of charge (Pease et al., 2007). The as-received solutions contained significant amounts of nonvolatile salts. The presence of high salt concentrations can interfere with the ES-DMA measurement in two ways. First, the evaporation of electrosprayed droplets containing high salt concentrations results in the formation of salt particles whose signal can overlap that of the antibody or other analytes of interest. Second, the salts can precipitate out on the surface of the antibody, encrusting it. The apparent size of these encrusted antibodies measured by the DMA then exceeds the actual size. Therefore, nonvolatile salts must be removed or largely reduced by dialyzing 150 μL of as-received antibodies with a slide-a-lyzer cartridge (Pierce, Rockford, Ill.) having a 10 kDa cutoff for 18 h in a 20 mM ammonium acetate solution at pH 8. All ammonium acetate solutions were prepared using deionized water (>18.0 MVcm) and adjusting the pH to 8.0 (with either glacial acetic acid or ammonium hydroxide). Upon removal of the antibodies from the dialysis cartridge, the volume had increased to 230 μL. Data for IgG antibodies were analyzed as reported in Pease et al. (Biotech & Bioeng, 2008).

### Example 4

### Ex vivo Fluorescent Experiments

Tissue specimens were collected from patients undergoing clinical upper endoscopy. Biopsies approximately 1-2 mm in diameter were taken from one or more places in the lower esophagus (5 cm proximal to the gastro-esophageal junction) and repeated 10 cm proximal to the initial site in the proximal esophagus. Tissue samples were taken via sterile forceps for frozen sectioning and for formaldehyde fixation. Samples were immediately wrapped in aluminum foil and placed in liquid nitrogen. After the sampling is complete, the samples were then transferred immediately to a −80° C. freezer where they were kept until sectioning. Alternatively, samples fixed in formaldehyde were embedded in paraffin. The samples were stored at −80° C. until use.

To demonstrate immunohistological localization of eosinophil granule proteins in EoE, formalin fixed tissues from 20 patients with EoE were stained using antibodies specific to EDN and MBP-1. Serial tissue sections were mounted on Superfrost Plus® slides (Fisher Scientific). The fixed sections were deparaffinized and incubated for one hour at 37° C. in 0.1% trypsin (Sigma, St. Louis, Mo.). The frozen tissue were cryosectioned. The slides were incubated in 10% normal mouse serum at 4° C. Serial sections were washed and overlaid with either control antibody (irrelevant murine monoclonal antibody or normal mouse serum) or monoclonal antibody to eosinophil granule proteins. Slides were washed and treated with 1% chromotrope 2R to eliminate nonspecific staining of eosinophils. Next, the slides were overlaid with fluorescein isothiocyanate-conjugated (FITC) rabbit anti-mouse IgG. Following a final wash, the sections were mounted in glycerol solution containing paraphenylenediamine as a retardant for fluorescent fading. Photomicrographic images were taken with a Zeiss Axiophot Microscope to document staining. Cellular and extracellular MBP-1 staining were graded on a visual analog scale of 0 to 4 with representative photomicrographs of each grade available for reference. The results showed localization of EDN and MBP-1 in EoE tissues. EDN is brilliantly localized on intact eosinophils, but is also deposited on and within membranes of esophageal epithelial cells. The observations confirm that marked EDN and EPO depositions are characteristic of EoE. For histology, the samples were fixed in 10% formalin for 24 h, dehydrated in ethanol and xylene, embedded in paraffin, stained with hematoxylin and eosin (H&E).

### Example 5

### Antibody Chelation, Radiolabeling, and Detection

Chelation of Antibodies.

The selected antibodies with irrelevant controls (murine immunoglobulins purchased commercially) can be chelated as described by Wang, et al. Nat Protoc, 2007. 2(4): p. 972-8. Antibodies can be concentrated to 10 mg/mL using Centricon spin columns. Buffer exchange can be used to remove any sodium azide used to temporarily preserve the antibodies. The concentration can be confirmed using the peak at 280 nm via UV-vis spectroscopy. A 0.3 M HEPES buffer at pH 7.6 can be prepared and refrigerated in small centrifuge vials. Deionized water can be purified to >12 MOhms and tested free from biologics. A small amount of NHS-MAG3 powder (0.24 mg) stored at −80° C. can be weighed on a scale to 0.01 mg and quickly transferred to protein nonbinding centrifuge vials. The NHS moiety of NHS-MAG3 binds to free amine groups on the antibody surface. Antibody (9.1 mg) can be added and vortexed before incubating at ambient temperature for 1-2 hr. The antibody can be separated from free NHS-MAG3 in a G25 column in 0.5 mL aliquots using 0.25 M ammonium acetate as an eluent. The concentration of antibody can then be confirmed using UV-vis.

Determination of Stoichiometries:

Quantifying stoichiometries can be used to determine how to enhance the signal from the radiolabeled antibody. Because UV-vis spectra overlap, the mAb-MAG3 ratio can be determined numerically by fitting spectra of chelated antibodies with a least squares fitting technique as a function of wavelength from 240 nm to 300 nm with the linear superposition of spectra of the mAb and NHS-MAG3, such that I₃=αI₁+βI₂ where I₁, I₂, and I₃ represent the intensities of the mAb, NHS-MAG3, and chelated mAb spectra. Beer's Law can be used to determine the ratio of concentrations from αI₁ (peak at 280 nm) and βI₂. The MAG3-Tc-99m ratio can be determined as follows: the activity of the Tc-99m can be correlated with its concentration using standard curves and activity measured using a dose calibrator. The MAG3 concentration can be determined using the mAb-MAG3 ratio and direct measurement of the protein concentration using UV-vis after 12 half-lives (˜72 hrs).

Radiolabeling:

The radionuclide Tc-99m can be added to the chelated antibodies using protocols. The radiolabeling procedure adopted from Wang, et al. Nat Protoc, 2007. 2(4): p. 972-8. can be used. For example, 40 ug of MAG3-protein conjugate can be added to 45 uL of 0.25 M ammonium acetate and 15 uL tartrate buffer and vortexed. Then 25 uL of Tc-99m and 5 uL of Sn solution (4 mg/mL of SnCl₂.2H₂O in tartrate buffer free of stannic chloride) can be added and gently vortexed before incubating for 1 hour at ambient temperature. The reduced Sn solution can be prepared freshly and temporarily stored under an N₂ environment. Binding efficacy can be assessed using a Sephadex G-25 (GE Lifesciences) column. The radioactivity of each aliquot and the column can then be measured using a dose calibrator and plotted.

Ex vivo Radiolabel Detection:

Tissue specimens collected previously can be cut into thin slices. These tissue slices can be from identically the same specimen as that used in the ex vivo fluorescent experiments The slices can be positioned on microscope slides with attention paid to tracking which portion of the surface faced the intraluminal surface. The slices can be first subjected to H&E and the eosinophil density can be determined.

A small aliquot of the radiolabeled antibody solution can be added to each slice. A reaction time is allowed. The antibody solution can be washed in triplicate in water or saline.

The dose is measured in dose calibrator as per above, in at least duplicate.

As a second method, the localized Tc-99m can determined using autoradiography. The samples can be placed above x-ray film. The system can then be covered to minimize inadvertent exposure and the radionuclide was allowed to decay. After 18 hours (approximately 3 half-lives), the samples can be removed and the x-ray film developed and transferred to digital images.

### Example 6

Antibody Gold Nanoparticle complex formation: An aliquot of human IgG antibodies was purchased from Sigma. Citrate stabilized gold nanoparticles 20 nm in diameter were purchased from Ted Pella. Antibodies and gold nanoparticles were combined in 1.5 mL microcentrifuge vials in varying ratios. The complex was electrosprayed and characterized using electrospray differential mobility analysis as described in Example 3. The size distribution, demonstrating binding, is shown in the accompanying figures. It is anticipated that the cystine residues on the surface of the antibody bind to the gold nanoparticles in a ligand exchange reaction with citrate.

### REFERENCES

Fogg M I, Ruchelli I E, Spergel J M. Pollen and eosinophilic esophagitis. J Allergy CLin Immunol 2003; 112(4):796-7.
Gonsalves N, Kahrilas P. Eosinophilic Esophagitis in Adults. Am J Gastroenterol 2009; epub.
Shah A, Kagawalla A F, Gonsalves N, et al. Histopathologic variability in Children with Eosinophilic Esophagitis. Am J Gastroenterol 2009; 104(3); 716-21.
Gonsalves N, Poliocarpio-Nicolas M, Zhang Q, Rao M A, Hirano I. Histopathologic variability and Endoscopic Correlates in Adults with Eosinophilic Esophagitis. Gastrointestinal Endosc 2006; 64(3):313-9.
Kephart G M, Alexander J A, Arora A S, Romero Y, Smyrk T C, Talley N J, Kita H. Marked deposition of eosinophil-derived neurotoxin in adult patients with eosinophilic esophagitis. Am J Gastroenterol. 2010 February; 105(2):298-307.
Chakrabarti M C et al., Prevention of radiolysis of monoclonal antibody during labeling, J. Nucl. Med., 1996, 37(8):1384-88.
Dawson, M., Krauland, E., Wirtz, D., Hanes, J., Transport of polymeric nanoparticle gene carriers in gastric mucus, Biotechnol Progr, 20 (2004) 851-857.
Gansow et al., Advanced Methods for Radiolabeling Monoclonal Antibodies with Therapeutic Radionuclides, Cancer Therapy with Radiolabeled Antibodies, 1995, Chapter 6, pp. 63-76.
Hnatowich et al., The Preparation of DTPA-Coupled Antibodies Radiolabeled with Metallic Radionuclides: an Improved Method, J. Immunological Methods, 1983, 65:147-157.
Lai, S. K., et al., Rapid transport of large polymeric nanoparticles in undiluted human cervical vaginal mucus. P Natl Acad Sci USA, 104 (2007) 1482-1487.
Lewis et al., A facile, water-soluble method for modification of proteins with DOTA, Bioconj Chem., 1994, 5:565-76.
Mackenzie, S. H., Go, M., Chadwick, B., Thomas, K., Fang, J., Kuwada, S., Lamphier, S., Hilden, K., Peterson, K. A., Eosinophilic esophagitis in patients presenting with dysphagia—a prospective analysis, Aliment Pharmacol Ther 28 (2008) 1140-1146.
Mansoor N. Saleh, et al., Clinical Use of a Standard Kit-Preparation of Radiolabeled Monoclonal, Antibody 96.5 in the Diagnostic Imaging of Metastatic Melanoma, J Clin Oncol 6:1059-1065 (1988).
Mirzadeh S., et al., Radiometal labelling of immunoproteins: Covalent linkage of 2-(4-Isothiocyanatobenzyl)diethylenetriaminepentaacetic acid ligands to immunoglobulin, Bioconj Chemistry, 1990, 1(1):59.
Mulder, D. J., Pacheco, I., Hurlbut, D. J., Mak, N., Furuta, G. T., MacLeod, R. J., Justinich, C. J., FGF9-induced proliferative response to eosinophilic inflammation in Esophagitis, Gut (2009) in press.
Odze, R. D., Pathology of eosinophilic esophagitis: What the clinician needs to know, Am J Gastroenterol 104 (2009) 485-490.
Pentiuk, S., Putnam, P. E., Collins, M. H., Rothenberg, M. E., Dissociation between symptoms and histological severity in pediatric eosinophilic Esophagitis, J Pediatr Gastroenterol Nutr., 48 (2009) 152-160.
Peterson, K. A., Degranulation in eosinophilic esophagitis: a differentiating marker of disease, in preparation.
Prasad, G. A., et al., Secular trends in the epidemiology and outcomes of eosinophilic esophagitis in Olmsted County, Minnesota (1976-2007), Digestive Disease Week, May 2008.
Richardson et al., Optimization and batch production of DTPA-labeled antibody kits for routine use in 111 in immunoscintography, Nuclear Medicine Communications, 1987, 8:346-356.
Swaminathan, G. J., Myszka, D. G., Katsamba, P. S., Ohnuki, L. E., Gleich, G. J., Acharya, K. R. Eosinophil-granule major basic protein, a C-type lectin, binds heparin, Biochem 44 (2005) 14152-8.
Wagner, L., Leiferman, K. M., Gleich, G. J., Eosinophils, Encyclopedia of Life Sciences, John Wiley & Sons, 2006.
Wasmoen, T. L. Bell, M. P. Loegering, D. A., Gleich, G. J., Prendergast, F. G., McKean, D. J., Biochemical and amino acid sequence analysis of human eosinophil granule major basic protein, J Biol Chem 263 (1988) 12559-12563.
G J Gleich, et al., Physiochemical and biological properties of the major basic protein from guinea pig eosinophil granules, J Exp Med 140 (1974) 313-332.
Prasad, Ganaphthy A. et al, “Secular Trends in the Epidemiology and Outcomes of Eosinophilic Esophagitis in Olmsted County, Minnesota (1976-2007).” Abstract, Digestive Disease Week, May 2008.
Mackenzie S, Go M, Thomas K, et al. Prospective Analysis of Eosinophilic Esophagitis in Patients Presenting with Dysphagia. Aliment Pharmacol Therapeutics 2008: in publication.
Liacouras C A, Spergel J M, Ruchelli E, et al. Eosinophilic Esophagitis: a 10-year experience in 381 children. Clin Gastroenterol hepatol 2005; 3:1198-1206.
Fogg M, Ruchelli E, Spergel J M. Pollen and Eosinophilic Esophagitis. J Allergy Clin Immunol 2003; 112:796-7.
Wang F Y, Gupta S K, Fitzgerald J F. Is there a seasonal variation in the incidence or intensity of allergic eosinophilic esophagitis in newly diagnosed children? Jo Clin Gastroenterol Hepatol 2007; 41:451-453.
Mishra A, Hogan S P, Brandt E B, Rothenberg M E. An etiological role for aeroallergens and eosinophils in experimental eosinophilic esophagitis. J Clin Invest 2001; 107: 83-90.
Konikoff et al. A Randomized, Double-blind, Placebo controlled trial of fluticasoneProprionate for Pediatric Eosinophilic Esophagitis. Gastroenterol 2006; 131:1381-91.
Markowitz J, Spergel J, Ruchelli E, Liacouras C. Elemental Diet is Effective Treatment for Eosinophilic Esophagitis in Children and Adolescents. Am J Gastroenterol 2003; 98:777-82.
Liacouras C A, Spergel J M, Ruchelli E, et al. Eosinophilic Esophagitis: a 10-year experience in 381 children. Clin Gastroenterol hepatol 2005; 3:1198-1206.
Kagawalla A F, Sentongo T A, Ritz S, et al. Effect of 6 food Elimination diet on clinical and histologic outcomes in eosinophilic esophagitis. Clin Gastroenterol Hepatol 2006; 4:1097-1102
Gonsalves N, Guang-Yu Y, Doerfler B, Ritz, S, Ditto A, Hirano I. A Prospective Trial of Six Food Elimination Diet and Reintroduction of Causative Agents in Adults with Eosinophilic Esophagitis. Digestive Disease Week Presentation 2008. No. 727
Gangotena F, Mackenzie S, Go M, et al. Eosinophilic Esophagitis, Ringed Esophagus: The Diagnostic Conundrum. Am J Gastroenterol 2007; 102: Abstract S79.
Gonsalves N, Duerfler B, Yang G Y, et al. Prospective Clinical Trial of Six Food Elimination Diet or Elemental Diet in the Treatment of Adults with Eosinophilic Esophagitis. S1181. Dig Dis Week 2009.
Gonsalves N, Kahrilas P. Eosinophilic Esophagitis in Adults. Am J Gastroenterol 2009; epub
Shah A, Kagawalla A F, Gonsalves N, et al. Histopathologic variability in Children with Eosinophilic Esophagitis. Am J Gastroenterol 2009; 104(3); 716-21.
Gonsalves N, Poliocarpio-Nicolas M, Zhang Q, Rao M A, Hirano I. Histopathologic variability and Endoscopic Correlates in Adults with Eosinophilic Esophagitis. Gastrointestinal Endosc 2006; 64(3):313-9.
Kephart G M, Alexander J A, Arora A S, Romero Y, Smyrk T C, Talley N J, Kita H. Marked deposition of eosinophil-derived neurotoxin in adult patients with eosinophilic esophagitis. Am J Gastroenterol. 2010 February; 105(2):298-307.
Kato M, Kephart G M, Talley N J, Wagner J M, Sarr M G, Bonno M, McGovern T W, Gleich G J: Eosinophil infiltration and degranulation in normal human tissue. Anat Record 252:418-425, 1998.
Peters M S, Rodriguez M, Gleich G J: Localization of human eosinophil granule major basic protein, eosinophil cationic protein, and eosinophil-derived neurotoxin by immunoelectron microscopy. Lab Invest 54:656-662, 1986.
Abu-Ghazaleh R I, Dunnette S L, Loegering D A, Checkel J L, Kita H, Thomas L L, Gleich G J: Eosinophil granule proteins in peripheral blood granulocytes. J Leukoc Biol 52:611-618, 1992.
Gleich G J, Loegering D A, Bell M P, Checkel J L, Ackerman S J, McKean D J: Biochemical and functional similarities between human eosinophil-derived neurotoxin and eosinophil cationic protein: homology with ribonuclease. Proc Natl Acad Sci USA 83:3146-3150, May, 1986.
O'Donnell M C, Ackerman S J, Gleich G J, Thomas L L: Activation of basophil and mast cell histamine release by eosinophil granule major basic protein. J Exp Med 157:1981-1991, June, 1983.
Gleich G J, Loegering D A, Mann K G, Maldonado J E: Comparative properties of the Charcot-Leyden crystal protein and the major basic protein from human eosinophils. J Clin Invest 57:633-640, March, 1976
Gleich G J, Loegering D A, Kueppers F, Bajaj S P, Mann K G: Physiochemical and biological properties of the major basic protein from guinea pig eosinophil granules. J Exp Med 140:313-332, August, 1974.
Frigas E, Loegering D A, Gleich G J: Cytotoxic effects of the guinea pig eosinophil major basic protein on tracheal epithelium. Lab Invest 42:35-43, 1980.
Gundel R H, Letts L G, Gleich G J: Human eosinophil major basic protein induces airway constriction and airway hyperresponsiveness in primates. J Clin Invest 87:1470-1473, April, 1991.

Throughout this application, various publications are referenced. The disclosures of these publications in their entireties are hereby incorporated by reference into this application in order to more fully describe the state of the art to which this invention pertains.

It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

1. A method of producing a three-dimensional map of an esophagus in a subject, comprising detecting an eosinophil protein in the lumen of the esophagus in a subject, comprising administering to the subject a labeled antibody directed against an eosinophil protein under conditions wherein the labeled antibody can bind to the eosinophil protein, and detecting a labeled antibody/eosinophil protein complex in the lumen of the esophagus, whereby detecting the labeled antibody/eosinophil protein complex in the lumen of the esophagus produces a three-dimensional map of the esophagus in the subject.

2. The method of claim 1, wherein the eosinophil protein is eosinophil surface proteins, eosinophil granule proteins, or secretory products of eosinophils.

3. The method of claim 2, wherein the eosinophil protein is CD9, Eotaxin-3, or CCR3.

4. The method of claim 2, wherein the eosinophil protein is major basic protein 1 (MBP-1), major basic protein 2 (MBP-2), eosinophil derived neurotoxin (EDN), Eosinophil cationic protein (ECP) or eosinophil peroxidase (EPO).

5. The method of claim 1, wherein the antibody is a monoclonal antibody or a polyclonal antibody.

6. The method of claim 1, wherein the antibody is radiolabeled.

7. The method of claim 6, wherein the radiolabel is Tc-99m or In-111.

8. The method of claim 1, wherein the labeled antibody is attached to a nanoparticle.

9. The method of claim 7, wherein the nanoparticle is made of gold, chitosan, titanium oxide, pluronic/lipiodol, or bismuth sulfide.

10. The method of claim 8, wherein the nanoparticle is a gold nanoparticle.

11. The method of claim 1, wherein the labeled antibody is administered to the subject orally.

12. The method of claim 1, wherein the labeled antibody is administered to the subject in an aerosol.

13. The method of claim 1, wherein the subject is a mammal.

14. The method of claim 13, wherein the mammal is a human.

15-28. (canceled)

29. A method of detecting a change in eosinophilic esophagitis in a subject diagnosed with eosinophilic esophagitis, comprising:
a) producing a first three-dimensional map of the esophagus in a subject diagnosed with eosinophilic esophagitis according to the method of claim 1;
b) producing a second three-dimensional map of the esophagus in the subject of step (a), according to the method of claim 1; and
c) comparing the three-dimensional map of step (b) with the three-dimensional map of step (a), whereby detecting a change in the three-dimensional map of step (b) compared to the three-dimensional map of step (a) detects a change in eosinophilic esophagitis in the subject.

30-52. (canceled)

53. The method of claim 44, wherein the antibody/eosinophil protein complex is detected by MRI analysis.

54. The method of claim 44, wherein the molecular map can be a two- or three-dimensional molecular map.

55-64. (canceled)

65. A method of detecting eosinophilic degranulation in a subject, comprising detecting a granule protein in a subject, comprising administering to the subject a labeled antibody directed against a granule protein under conditions wherein the labeled antibody can bind to the granule protein, and detecting a labeled antibody/granule protein complex, whereby detecting the labeled antibody/granule protein complex indicates the presence of eosinophilic degranulation in the subject.

66. (canceled)

67. The method of claim 65, further comprising detecting eosinophils in the subject, comprising detecting an eosinophilic protein in a subject, comprising administering to the subject a labeled antibody directed against an eosinophilic protein under conditions wherein the labeled antibody can bind to the eosinophilic protein, and detecting a labeled antibody/eosinophilic protein complex, whereby detecting the labeled antibody/eosinophilic protein complex indicates the presence of eosinophils in the subject.

68. The method of claim 65, wherein the radiolabel for the labeled antibody directed against a granule protein and the label for the labeled antibody directed against an eosinophilic protein are different.

69. The method of claim 1, wherein the three dimensional map of the esophagus is used to diagnose eosinophilic esophagitis in a subject.

70. The method of claim 44, wherein the molecular map of the esophagus is used to diagnose eosinophilic esophagitis in a subject.
